(19) United States
(12) Patent Application Publication
(10) Pub. No.: US 2011/0197305 A1
(43) Pub. Date: Aug. 11, 2011

(54) DENSE AND ERECT PANICLE GENE AND USES THEREOF

(76) Inventors: Xiangdong Fu, Beijing (CN); Xianzhong Huang, Beijing (CN); Qian Qian, Beijing (CN); Zhengbin Liu, Beijing (CN)

(21) Appl. No.: 12/996,147

(22) PCT Filed: Jun. 5, 2009
(86) PCT No.: PCT/IB09/06658

§ 371 Date: Mar. 30, 2011

(30) Foreign Application Priority Data
Jun. 5, 2008 (CN) 200810111529.5

(51) Int. Cl. A01H 1/00; C07H 21/04; C12N 15/63; C12N 5/10; C12N 1/00; A01H 5/00; A01H 5/10

(52) U.S. Cl. 800/275; 536/23.6; 435/320.1; 435/419; 435/325; 435/243; 800/295; 800/298; 800/320.1; 800/320.3; 800/320; 800/317.2; 800/313; 800/306; 800/312; 800/317; 800/309; 800/310; 800/315; 800/316; 800/317.4; 800/314; 800/322; 800/307; 800/305; 800/278; 530/300; 800/260

(57) ABSTRACT

Compositions and methods for imparting a dense and erect panicle phenotype to plants, including polynucleotides, polypeptides, vectors and cells. This phenotype is associated with improving plant traits, such as improving plant yield.

### CROSS-REFERENCE TO RELATED APPLICATIONS

The present application claims priority to Chinese Patent Application No. 200810111529.5 filed 5 Jun. 2008.

### TECHNICAL FIELD OF THE INVENTION

The invention relates generally to compositions and methods for imparting a dense and erect panicle phenotype to plants, including polynucleotides, polypeptides, vectors and host cells. This phenotype is associated with improving plant traits, such as improving plant yield. The present invention also relates generally to plants transformed by the aforementioned compositions and methods.

### BACKGROUND OF THE INVENTION

Rice (Oryza sativa) is one of mankind's major food staples. Given continuing population growth and increasing competition for arable land between food and energy crops, food security is becoming an ever more serious global problem. Improving crop productivity by selection for the components of grain yield and for optimal plant architecture has been the key focus of national and international rice breeding programs.

In the 1960s, a high-yield semi-dwarf variety of rice known as IR8 was developed, which profoundly revolutionized rice breeding. However, limitations in IR8 and related varieties caused plant breeders and physiologists at the International Rice Research Institute (IRRI) to postulate that a new plant type (or ideotype) needed to be developed to meet future needs. Accordingly, in 1989 the IRRI issued a strategic plan to develop a new plant type with a yield potential 20-25% higher than that of existing semi-dwarf varieties of rice. The proposed new plant type possessed an increased height, a low tillering capacity with fewer unproductive tillers, an earbearing tiller percentage increase, larger panicles with more grains per panicle, a vigorous root system, and improvement in both biomass and economic coefficient.

In the late 1980s, different, but similarly advantageous, ideotypes were proposed in China. These ideotypes were based upon erect panicle rice varieties that first appeared in the 1930s, developed in the 1960s, and popularized in the 1980s. The erect panicle varieties present in China are derived from the main cultural variety “Balilla” of Italy, and some important varieties include “Liaojing 5#”, “Qianchonglang”, “Shennong 265”, and “Shennong 606”. Erect panicle rice varieties are currently dominant in northeastern China, and are significant contributors to overall rice production and research in that nation.

These ideotypes were proposed because, as compared to a curved panicle, an erect or semi-erect panicle has many advantages. Erect panicles are more efficient in utilizing light energy and are superior to curved panicles with respect to environmental conditions required to produce the same yield (e.g., illumination, temperature, humidity, gas diffusion). Plants with erect panicles also have a higher growth rate and produce greater amounts of dry matter, both of which increase yield.

The dense and erect panicle phenotype is usually associated with dwarfism, which improves plant shape and the balance of yield-associated factors—in particular, both panicle number and grain number per panicle. The dense and erect panicle phenotype is also significantly superior to the curved panicle phenotype in lodging resistance, because an erect panicle has a significantly lower acting force of panicle to stalk than that of a curved panicle. The dense and erect panicle phenotype also has short and thick basal internodes, a leaf sheath with a high bearing capacity, greater matter production, and a decreased transfer amount to grains after earing.

At present, few studies have been directed to the gene(s) responsible for the erect panicle phenotype. It was speculated that this phenotype was controlled by a single recessive nuclear gene. Others postulated that this phenotype was controlled by a pair of nuclear genes or a pair of additive genes. Still others reported that the gene responsible for the erect panicle phenotype was located on chromosome 9 between two SSR (simple-sequence repeat) markers, RM5833-11 and RM5686-23, at a genetic distance of 1.5 and 0.9 cM, respectively. It was also reported that a major QTL controlling eject panicle gene, qEP9-1, was located on chromosome 9 between STS marker H90 and SSR marker RM5652.

However, prior to the disclosure of the present invention, the gene(s) responsible for this ideotype remained otherwise unidentified and had yet to be isolated. In view of the aforementioned advantages demonstrated by the dense and erect panicle phenotype in addressing the continuing unmet need to produce higher-yield rice and other crops, the identification and isolation of this gene or genes is of great importance.

### BRIEF SUMMARY OF THE INVENTION

The present invention relates to isolated DEP1 polynucleotides, polypeptides, vectors and host cells expressing isolated DEP1 polynucleotides capable of imparting the dense and erect panicle phenotype to plants, including rice. The related polynucleotides, polypeptides, vectors and cells of the present invention are also capable of imparting specific traits to plants, and in particular crop plants. These traits include increased yield, increased lodging resistance, increased panicle number, increased grain number per panicle, dwarf or semi-dwarf stature, increased photosynthetic efficiency, increased population growth rate during grain filling period, increased water transport capacity, increased mechanical strength of the stem, and increased dry matter production.

The isolated DEP1 polynucleotides provided herein include nucleic acids comprising (a) a nucleotide sequence of any one of SEQ ID NOs: 1 and 5-8; (b) a nucleotide sequence at least 70% identical to (a); (c) those that specifically hybridize to the complement of (a) under stringent hybridization conditions; (d) an open reading frame encoding a DEP1 protein comprising a polypeptide sequence of any one of SEQ ID NOs: 9 and 11-14; (e) an open reading frame encoding a DEP1 protein comprising a polypeptide sequence at least 70% identical to any one of SEQ ID NOs: 9 and 11-14; and (f) a nucleotide sequence that is the complement of any one of (a)-(e). The isolated polynucleotides provided herein also include nucleic acids comprising (a) a nucleotide sequence of SEQ ID NO: 2; (b) a nucleotide sequence at least 70% identical to (a); (c) those that specifically hybridize to the complement of (a) under stringent hybridization conditions; (d) an open reading frame encoding a DEP1 protein comprising a polypeptide sequence of SEQ ID NO: 10; (e) an open reading frame encoding a DEP1 protein comprising a polypeptide sequence at least 70% identical to SEQ ID NO: 10; and (f) a nucleotide sequence that is the complement of any one of (a)-(e). The isolated polynucleotides provided herein also include sequences having promoter function. These sequences include (a) a nucleic acid comprising a nucleotide sequence of SEQ ID NO: 4; (b) a nucleic acid comprising a nucleotide sequence at least 70% identical to (a); (c) a nucleic acid that specifically hybridizes to the complement of (a) under stringent hybridization conditions; and (d) a nucleotide sequence that is the complement of any one of (a)-(c).

The isolated DEP1 polypeptides provided herein include (a) an amino acid sequence of any one of SEQ ID NOs: 9 and 11-14; and (b) an amino acid sequence at least 70% identical to (a). Also included are polypeptides comprising (a) an amino acid sequence of SEQ ID NO: 10; and (b) an amino acid sequence at least 70% identical to (a). Also included are isolated polypeptides comprising amino acid sequences of any one of SEQ ID NOs: 30-33.

The host cells provided herein include those comprising the isolated polynucleotides and vectors of the present invention. The host cell can be from an animal, plant, or microorganism, such as E. coli. Plant cells are particularly contemplated. The host cell can be isolated, excised, or cultivated. The host cell may also be part of a plant.

The present invention further relates to a plant or a part of a plant that comprises a host cell of the present invention. Rice, wheat, barley, maize, oat, soybean and rye are particularly contemplated. The present invention also relates to the transgenic seeds of the plants.

The present invention further relates to a method for producing a plant comprising regenerating a transgenic plant from a host cell of the present invention, or hybridizing a transgenic plant of the present invention to another non-transgenic plant. Plants produced by these methods are also encompassed by the present invention, and plants having a dense and erect panicle phenotype are particularly contemplated, as are crop plants, such as rice, wheat, barley, maize, oat, soybean and rye.

The present invention further relates to methods of altering a trait in a plant or part of a plant using the isolated polynucleotides, polypeptides, constructs and vectors of the present invention. These traits include yield, lodging resistance, panicle number, grain number per panicle, dwarf or semi-dwarf stature, photosynthetic efficiency, population growth rate during grain filling period, water transport capacity, mechanical strength of the stem, and dry matter production. Preferably these traits are altered so that they are increased or otherwise improved. In one embodiment, these traits are increased or improved by reducing the expression of DEP1 nucleic acids or proteins, such as SEQ ID NOs: 2 and 10. In another embodiment, these traits are increased or improved by expressing a mutant DEP1 nucleic acid or protein (i.e., dep1) in the plant, such as SEQ ID NOs: 1, 5-8, 9, and 11-14.

The present invention further relates to the use of the isolated polynucleotides, polypeptides, constructs and vectors of the present invention to alter plant traits, e.g., yield, lodging resistance, panicle number, grain number per panicle, dwarf or semi-dwarf stature, photosynthetic efficiency, population growth rate during grain filling period, water transport capacity, mechanical strength of the stem, and dry matter production. Preferably these traits are altered so that they are increased or otherwise improved. In one embodiment, these traits are increased or improved by reducing the expression of DEP1 nucleic acids or proteins, such as SEQ ID NOs: 2 and 10. In another embodiment, these traits are increased or improved by expressing in the plant a mutant DEP1 gene or protein (i.e., dep1), such as SEQ ID NOs: 1, 5-8, 9, and 11-14.

The present invention further relates to methods of identifying DEP1 binding agents and inhibitors. In one embodiment, the method comprises (a) providing an isolated DEP1 protein; (b) contacting the isolated DEP1 protein with an agent under conditions sufficient for binding; (c) assaying binding of the agent to the isolated DEP1 protein; and (d) selecting an agent that demonstrates specific binding to the isolated DEP1 protein.

In another embodiment, the method comprises (a) providing a host cell expressing a DEP1 protein; (b) contacting the host cell with an agent; (c) assaying expression of DEP1 protein; and (d) selecting an agent that induces altered expression of DEP1 protein. In certain embodiments, e.g., when the host cell expresses a full-length DEP1, such as SEQ ID NO: 10, an agent is selected that reduces expression of the protein. In other embodiments, e.g., when the host cell expresses a truncated DEP1 protein, such as SEQ ID NO: 9, an agent is selected that increases expression of the protein.

In another embodiment, the method comprises (a) providing a plant or part of a plant expressing a DEP1 protein; (b) contacting the plant or the part of the plant with an agent; (c) assaying for alteration of a trait of the plant or the part of the plant; and (d) selecting an agent that alters the trait. The traits to be assayed are those known to be affected by DEP1 expression (e.g., yield, lodging resistance, panicle number, grain number per panicle, dwarf or semi-dwarf stature, photosynthetic efficiency, population growth rate during grain filling period, water transport capacity, mechanical strength of the stem, and dry matter production). Preferably agents that increase or otherwise improve these traits are selected. However, agents that negatively impact a trait are contemplated as well.

The present invention also relates to methods of inhibiting DEP1 in a plant using the binding agents and inhibitors identified by the methods herein.

### BRIEF SUMMARY OF THE SEVERAL VIEWS OF THE DRAWINGS

FIG. 1 shows photos of a typical whole plant of Shao 313 (dense and erect panicle, see the right side) and Shao 314 (curved and loose panicle, see the left side).

FIG. 2 shows the sequence analysis of the DEP1 and dep1 gene products. (a) Alignment of dep1 with DEP1. (b) Alignment of the putative PEBP-like domain with the N-terminus of the GS3 protein (SEQ ID NO:29). The numbers on the right indicate the position of the residues in the full protein. Identical and conserved residues indicated by dark grey boxes, and variant residues by light grey boxes. (c) Allelic variation of the DEP1 sequence.

FIG. 3 shows the phenotype of NIL-dep1 plants. (a) Dense and erect panicle. Scale bar, 4 cm. (b) Increased panicle branching and reduced rachis length. Scale bar, 4 cm. (c-i) Comparison of panicle architecture. (c) Number of grains per panicle. (d) Number of culms. (e) Panicle length. (f) Number of primary branches per panicle. (g) Number of secondary branches per panicle. (h) 1,000-grain weight. (i) Grain yield per plant. The NIL plants were grown in standard paddy field with a distance of 15×15 cm under conventional cultivation conditions. All data are given as mean±s.e.m. (n=36 plants). A Student's t-test was used to generate the P values.

FIG. 4 illustrates the differences in photosynthesis between the two lines.

FIG. 5 illustrates the differences in chlorophyll content between the two lines.

FIG. 6 illustrates the differences in stem vascular bundle number and midrib vascular bundle number between the two lines.

FIG. 7 shows the differences in vascular bundles between NIL-dep1 plants and NIL-DEP1 plants. (a) The internodes of NIL-dep1 and NIL-DEP1 plants. (b) The increased number of vascular bundles in the flag leaf veins of NIL-dep1 plants.

FIG. 8 shows the results of complementary transgenic verification studies. (a) The reduced expression of dep1 induces changes in panicle architecture in NIL-dep1 plants carrying pDEP1:RNAi-DEP1. Scale bar: 2 cm. (b) The panicle architecture of non-transgenic and transgenic NIL-DEP1 plants carrying the pDEP1:dep1 construct. Scale bar: 3 cm. (c) The numbers of grains per main panicle is higher in transgenic NIL-DEP1 plants expressing dep1 under the control of the native DEP1 promoter. Data given as mean±standard error (n=30 plants). (d) Transgenic NIL-DEP1 plants expressing DEP1 under the control of the native DEP1 promoter does not alter panicle architecture. Scale bar: 3 cm. (e), Transgenic Nipponbare plants constitutively expressing dep1 under the control of the rice actin1 promoter have a dwarf stature. Scale bar: 10 cm. (f) The structure of the main panicle of transgenic Nipponbare plants expressing DEP1 driven by the rice actin1 promoter. Scale bar: 4 cm.

FIG. 9 shows a typical result of overexpression study, in which, from left to right, the 1st panicle is non-transgenic Nipponbare control, and the 2nd to 4th panicles are dep1 transgenic Nipponbare.

FIG. 10 shows the panicle architecture of a dep1 NIL in the background of indica variety ZF 802. (a) Panicle characterization of ZF 802 (dep1). Scale bar: 2 cm. (b) Mature de-seeded panicles showing rachis length and panicle branching. Scale bar: 2 cm. (c) Number of grains per panicle in ZF 802 (dep1) and wild type ZF 802. Data given as mean±standard error (n=20 plants).

FIG. 11 shows dep1 expression in different transgenic plants by RT-PCR analysis, in which NP represents Nipponbare, 1-7 represent different transgenic Nipponbare plants with overexpression of pAct::dep1.

FIG. 12 shows the expression of dep1 in various organs and different stages of inflorescences development. C, culm; R, root; LB, leaf blade; LS, leaf sheath; SAM, shoot apex meristem; RM, rachis meristem; BM, branch meristem; SM, spikelet meristem; FL, floral meristem. Rice actin1 was used as a control.

FIG. 13 shows the allelic variation for DEP1 in domesticated and wild rice. The numbers on the right indicate the position of residues in the full length protein. The japonica varieties represented are Nipponbare, Wanhui 31 (WH 3), Shao 313; and the indica varieties are Guangluai 4 (GLA4), Zheshan 97B (ZX97B), TN 1, 93-11, Nanjing 6 (NJ 6), Zhefu 802 (ZF 802), Minghui 63 (MH 63), Miyang 46 (MY 46), Peiai 64 (PA 64), Teqing; the accession of wild rice (O. rufipogon) is Dongxiang wild rice. Dongxiang wild rice, Nipponbare, Wanhui 31 (WH 3) and Shao 314 all express the same version of the DEP1 protein (SEQ ID NO:10). As described elsewhere, Shao 313 express a truncated protein, dept (SEQ ID NO: 9). Guangluai 4 (GLA4), Zheshan 97B (ZX97B), 93-11 and Minghui 63 (MH 63) express a slightly different full-length DEP1 (SEQ ID NO: 30). TN 1, Nanjing 6 (NJ 6) and Zhefu 802 also express a slightly different full-length DEP1 (SEQ ID NO: 31). Miyang 46 (MY 46), Peiai 64 (PA 64), and Teqing express yet another slightly different full-length DEP1 (SEQ ID NO: 32).

FIG. 14 shows a phylogenetic analysis of DEP1 homologs among the small-grained cereals, in which identical and conserved residues are indicated by dark gray boxes and variant residues by light gray boxes. TaDEP1 (SEQ ID NO: 11) is the protein expressed in the bread wheat variety Ni982105 (Triticum aestivum). HvDEP1 (SEQ ID NO: 12) is the protein expressed in barley (Hordeum vulgare). TuDEP1 SEQ ID NO: 33) is the protein expressed in the bread wheat diploid wild progenitor (Triticum urartu).

FIG. 15 shows the phenotype observed by overexpressing the wheat and barley homogenous DEP1 gene in Nipponbare rice. a) Panicle phenotype transformed with wheat TaDEP1 gene; b) grain number per panicle of plant transformed with wheat TaDEP1 gene; c) panicle phenotype transformed with barley HvDEP1 gene; d) grain number per panicle of plant transformed with barley HvDEP1 gene. In each of a) to d), the left panel represents transgenic recipient plant and the right panel represents transgenic positive plant.

FIG. 16 is a comparison of ear length and structure between transgenic wheat plants carrying pUbi-RNAi-TaDEP1 and wild-type controls. Scale bar, 7 cm. Data are given as mean±s.e.m. (n=20 plants).

FIG. 17 is the dep1 cDNA sequence isolated from Shao 313 (SEQ ID NO: 1).

FIG. 18 is the DEP1 cDNA sequence isolated from Shao 314 (SEQ ID NO: 2).

FIGS. 19a and 19b are the DEP1 gDNA sequence isolated from Shao 314 (SEQ ID NO: 3).

FIG. 20 is the dep1 promoter sequence isolated from Shao 313 (SEQ ID NO: 4).

FIG. 21 is the dep1 homolog cDNA sequence from wheat (SEQ ID NO: 5).

FIG. 22 is the dep1 homolog cDNA sequence from barley (SEQ ID NO: 6).

FIG. 23 is a first dep1 homolog cDNA sequence from maize (SEQ ID NO: 7).

FIG. 24 is a second dep1 homolog cDNA sequence from maize (SEQ ID NO: 8).

FIG. 25 is the dep1 protein sequence from Shao 313 (SEQ ID NO: 9).

FIG. 26 is the DEP1 protein sequence from Shao 314 (SEQ ID NO: 10).

FIG. 27 is the wheat homolog protein sequence (SEQ ID NO: 11).

FIG. 28 is the barley homolog protein sequence (SEQ ID NO: 12).

FIG. 29 is the first maize homolog protein sequence (SEQ ID NO: 13).

FIG. 30 is the second maize homolog protein sequence (SEQ ID NO: 14).

### DETAILED DESCRIPTION OF THE INVENTION

### DEP1 Nucleic Acids and Proteins

As used herein, the terms “nucleic acid”, “polynucleotide”, “polynucleotide molecule”, “polynucleotide sequence” and plural variants are used interchangeably to refer to a wide variety of molecules, including single strand and double strand DNA and RNA molecules, cDNA sequences, genomic DNA sequences of exons and introns, chemically synthesized DNA and RNA sequences, and sense strands and corresponding antisense strands. Polynucleotides of the invention may also comprise known analogs of natural nucleotides that have similar properties as the reference natural nucleic acid.

As used herein, the terms “polypeptide”, “protein” and plural variants are used interchangeably and refer to a compound made up of a single chain of amino acids joined by peptide bonds. Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Exemplary DEP1 polynucleotides of the invention are set forth as SEQ ID NOs: 1-3 and 5-8 and substantially identical sequences encoding DEP1 proteins capable of altering a trait of a plant, for example, improving yield, improving lodging resistance, improving panicle number, improving grain number per panicle, dwarf or semi-dwarf stature, improving photosynthetic efficiency, improving population growth rate during grain filling period, improving water transport capacity, improving mechanical strength of the stem, and improving dry matter production.

Exemplary DEP1 polypeptides of the invention are set forth as SEQ ID NOs: 9-14 and substantially identical proteins capable of altering a trait of a plant, for example, improving yield, improving lodging resistance, improving panicle number, improving grain number per panicle, dwarf or semi-dwarf stature, improving photosynthetic efficiency, improving population growth rate during grain filling period, improving water transport capacity, improving mechanical strength of the stem, and improving dry matter production.

Substantially identical sequences are those that have at least 60%, preferably at least 80%, preferably at least 85%, more preferably at least 90%, even more preferably at least 95%, and most preferably at least 99% nucleotide or amino acid residue identity, when compared and aligned for maximum correspondence using a sequence comparison algorithm or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acids or proteins perform substantially the same function. Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues. Substantially identical nucleic acids are also identified as nucleic acids that hybridize specifically to or hybridize substantially to a reference sequence (e.g., SEQ ID NO: 1).

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Natl. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, Ausubel et al., infra).

One example of an algorithm that is suitable for determining percent sequence identity and sequence similarity is the BLAST algorithm, which is described in Altschul et al., J. Mol. Biol., 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA, 89:10915 (1989)).

In addition to calculating percent sequence identity, the BLAST algorithm also performs a statistical analysis of the similarity between two sequences (see e.g., Karlin & Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues.

Another indication that two nucleic acid sequences are substantially identical is that the two molecules hybridize to each other under stringent conditions. Stringent conditions are those under which a nucleic acid probe will typically hybridize to its target sequence but to no other sequences when that sequence is present in a complex nucleic acid mixture (e.g., total cellular DNA or RNA). Stringent hybridization conditions and stringent hybridization wash conditions in the context of nucleic acid hybridization experiments such as Southern and Northern blot analyses are both sequence- and environment-dependent. An extensive guide to the hybridization of nucleic acids is found in Tijssen, Laboratory Techniques in Biochemistry and Molecular Biology—Hybridization with Nucleic Acid Probes, part I chapter 2, Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. Another example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An exemplary medium stringency wash for a duplex of, e.g., more than 100 nucleotides, is 1×SSC at 45° C. for 15 minutes. An example low stringency wash for a duplex of, e.g., more than 100 nucleotides, is 4×-6×SSC at 40° C. for 15 minutes. For short probes (e.g., about 10 to 50 nucleotides), stringent conditions typically involve salt concentrations of less than about 1.0 M sodium ions, typically about 0.01 to 1.0 M sodium ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of hybridization and wash conditions that may be used to identify nucleotide sequences that are substantially identical to reference nucleotide sequences of the present invention. A substantially identical nucleotide sequence preferably hybridizes to a reference nucleotide sequence in 7% sodium dodecyl sulfate (SDS), 0.5 M NaPO₄, 1 mM EDTA at 50° C. with washing in 2×SSC, 0.1% SDS at 50° C., more preferably in 7% sodium dodecyl sulfate (SDS), 0.5 M NaPO₄, 1 mM EDTA at 50° C. with washing in 1×SSC, 0.1% SDS at 50° C., still more preferably in 7% sodium dodecyl sulfate (SDS), 0.5 M NaPO₄, 1 mM EDTA at 50° C. with washing in 0.5×SSC, 0.1% SDS at 50° C., even more preferably in 7% sodium dodecyl sulfate (SDS), 0.5 M NaPO₄, 1 mM EDTA at 50° C. with washing in 0.1×SSC, 0.1% SDS at 50° C., and most preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the that proteins encoded by the nucleic acids are substantially identical, share an overall three-dimensional structure, are biologically functional equivalents, or are immunologically cross-reactive with, or specifically bind to, each other. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code. This also includes degenerate codon substitutions wherein the third position of one or more selected (or all) codons is substituted with mixed-base and/or deoxyinosine residues (see Batzer et al., Nucleic Acids Res., 19:5081 (1991); Ohtsuka et al., J. Biol. Chem., 260:2605-2608 (1985); and Rossolini et al. Mol. Cell. Probes, 8:91-98 (1994)). However, both the polynucleotides and the polypeptides of the present invention may be conservatively substituted at one or more residues. Examples of conservative amino acid substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Nucleic acids of the invention also comprise nucleic acids complementary to SEQ ID NOs: 1-3 and 5-8, and subsequences and elongated sequences of SEQ ID NOs: 1-3 and 5-8 and complementary sequences thereof. Complementary sequences are two nucleotide sequences that comprise antiparallel nucleotide sequences capable of pairing with one another upon formation of hydrogen bonds between base pairs. As used herein, the term Like other polynucleotides in accordance with the present invention, complementary sequences maybe substantially similar to one another as described previously. A particular example of a complementary nucleic acid segment is an antisense oligonucleotide.

A subsequence is a sequence of nucleic acids that comprises a part of a longer nucleic acid sequence. An exemplary subsequence is a probe or a primer. An elongated sequence is one in which nucleotides (or other analogous molecules) are added to a nucleic acid sequence. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, introns, additional restriction enzyme sites, multiple cloning sites, and other coding segments. Thus, the present invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Polynucleotides of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art (see e.g., Sambrook et al. (eds.) Molecular Cloning: A Laboratory Manual, 1989, Cold Spring Harbor Laboratory Press, Cold Spring Harbor, N.Y.; Silhavy et al., Experiments with Gene Fusions, 1984, Cold Spring Harbor Laboratory Press, Cold Spring Harbor, N.Y.; Glover & Hames, DNA Cloning: A Practical Approach, 2nd ed., 1995, IRL Press at Oxford University Press, Oxford/New York; Ausubel (ed.) Short Protocols in Molecular Biology, 3rd ed., 1995, Wiley, New York).

Isolated polypeptides of the invention may be purified and characterized using a variety of standard techniques that are known to the skilled artisan (see e.g., Schröder et al., The Peptides, 1965, Academic Press, New York; Bodanszky, Principles of Peptide Synthesis, 2nd rev. ed. 1993, Springer-Verlag, Berlin/New York; Ausubel (ed.), Short Protocols in Molecular Biology, 3rd ed., 1995, Wiley, New York).

The present invention also encompasses methods for detecting a nucleic acid molecule that encodes a DEP1 protein. Such methods may be used to detect DEP1 gene variants or altered gene expression. Sequences detected by methods of the invention may detected, subcloned, sequenced, and further evaluated by any measure well known in the art using any method usually applied to the detection of a specific DNA sequence. Thus, the nucleic acids of the present invention may be used to clone genes and genomic DNA comprising the disclosed sequences. Alternatively, the nucleic acids of the present invention may be used to clone genes and genomic DNA of related sequences. Levels of a DEP1 nucleic acid molecule may be measured, for example, using an RT-PCR assay (see e.g., Chiang, J. Chromatogr. A., 806:209-218 (1998) and references cited therein).

The present invention also encompasses genetic assays using DEP1 nucleic acids for quantitative trait loci (QTL) analysis and to screen for genetic variants, for example by allele-specific oligonucleotide (ASO) probe analysis (Conner et al., Proc. Natl. Acad. Sci. USA, 80(1):278-282 (1983)), oligonucleotide ligation assays (OLAs) (Nickerson et al., Proc. Natl. Acad. Sci. USA, 87(22):8923-8927 (1990)), single-strand conformation polymorphism (SSCP) analysis (Orita et al., Proc. Natl. Acad. Sci. USA, 86(8):2766-2770 (1989)), SSCP/heteroduplex analysis, enzyme mismatch cleavage, direct sequence analysis of amplified exons (Kestila et al., Mol. Cell, 1(4):575-582 (1998); Yuan et al., Hum. Mutat., 14(5):440-446 (1999)), allele-specific hybridization (Stoneking et al., Am. J. Hum. Genet., 48(2):370-382 (1991)), and restriction analysis of amplified genomic DNA containing the specific mutation. Automated methods may also be applied to large-scale characterization of single nucleotide polymorphisms (Wang et al., Am. J. Physiol., 1998, 274(4 Pt 2):H1132-1140 (1992); Brookes, Gene, 234(2):177-186 (1999)). Preferred detection methods are non-electrophoretic, including, for example, the TAQMAN™ allelic discrimination assay, PCR-OLA, molecular beacons, padlock probes, and well fluorescence (see Landegren et al., Genome Res., 8:769-776 (1998) and references cited therein).

The present invention also encompasses functional fragments of a DEP1 polypeptide, for example, fragments that have the ability to alter a plant trait similar to that of any of SEQ ID NOs: 9-14. Functional polypeptide sequences that are longer than the disclosed sequences are also encompassed. For example, one or more amino acids may be added to the N-terminus or C-terminus of an antibody polypeptide. Such additional amino acids may be employed in a variety of applications, including but not limited to purification applications. Methods of preparing elongated proteins are known in the art.

The present invention also encompasses methods for detecting a DEP1 polypeptide. Such methods can be used, for example, to determine levels of DEP1 protein expression and correlate the level of expression with the presence or change in phenotype, trait, or level of expression in a different gene or gene product. In certain embodiments, the method involves an immunochemical reaction with an antibody that specifically recognizes a DEP1 protein. Techniques for detecting such antibody-antigen conjugates or complexes are known in the art and include but are not limited to centrifugation, affinity chromatography and other immunochemical methods (see e.g., Ishikawa Ultrasensitive and Rapid Enzyme Immunoassay, 1999, Elsevier, Amsterdam/New York, United States of America; Law, Immunoassay: A Practical Guide, 1996, Taylor & Francis, London/Bristol, Pa., United States of America; Liddell et al., Antibody Technology, 1995, Bios Scientific Publishers, Oxford, United Kingdom; and references cited therein).

### DEP1 Expression Systems

An expression system refers to a host cell comprising a heterologous nucleic acid and the protein encoded by the heterologous nucleic acid. For example, a heterologous expression system may comprise a host cell transfected with a construct comprising a DEP1 nucleic acid encoding a DEP1 protein operatively linked to a promoter, or a cell line produced by introduction of DEP1 nucleic acids into a host cell genome. The expression system may further comprise one or more additional heterologous nucleic acids relevant to DEP1 function, such as targets of DEP1 transcriptional activation or repression activity. These additional nucleic acids may be expressed as a single construct or multiple constructs.

A construct for expressing a DEP1 protein may include a vector sequence and a DEP1 nucleotide sequence, wherein the DEP1 nucleotide sequence is operatively linked to a promoter sequence. A construct for recombinant DEP1 expression may also comprise transcription termination signals and sequences required for proper translation of the nucleotide sequence. Preparation of an expression construct, including addition of translation and termination signal sequences, is known to one skilled in the art.

The promoter may be any polynucleotide sequence which shows transcriptional activity in the chosen plant cells, plant parts, or plants. The promoter may be native or analogous, or foreign or heterologous, to the plant host and/or to the DNA sequence of the invention. Where the promoter is native or endogenous to the plant host, it is intended that the promoter is found in the native plant into which the promoter is introduced. Where the promoter is foreign or heterologous to the DNA sequence of the invention, the promoter is not the native or naturally occurring promoter for the operably linked DNA sequence of the invention. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley et al., Nucleic Acids Res., 15:2343-61 (1987). Also, the location of the promoter relative to the transcription start may be optimized (see e.g., Roberts et al., Proc. Natl. Acad. Sci. USA, 76:760-4 (1979)). Many suitable promoters for use in plants are well known in the art. An exemplary promoter suitable for use with the present invention is set forth in SEQ ID NO:4.

For example, suitable constitutive promoters for use in plants include the promoters from plant viruses, such as the peanut chlorotic streak caulimovirus (PClSV) promoter (U.S. Pat. No. 5,850,019); the 35S and 19S promoters from cauliflower mosaic virus (CaMV) (Odell et al., Nature, 313:810-812 (1985) and U.S. Pat. No. 5,352,605); the promoters of Chlorella virus methyltransferase genes (U.S. Pat. No. 5,563,328) and the full-length transcript promoter from figwort mosaic virus (FMV) (U.S. Pat. No. 5,378,619); the promoters from such genes as rice actin (McElroy et al., Plant Cell, 2:163-171 (1990)); ubiquitin (Binet et al., Plant Science, 79:87-94 (1991)), maize (Christensen et al., Plant Molec. Biol., 12: 619-632 (1989)), and arabidopsis (Norris et al., Plant Molec. Biol., 21:895-906 (1993); and Christensen et al., Plant Mol. Biol., 18:675-689 (1982)); pEMU (Last et al., Theor. Appl. Genet., 81:581-588 (1991)); MAS (Velten et al., EMBO J., 3:2723-2730 (1984)); maize H3 histone (Lepetit et al., Mol. Gen. Genet., 1992, 231:276-285 (1992); and Atanassova et al., Plant J., 2(3):291-300 (1992)); Brassica napus ALS3 (PCT International Publication No. WO 97/41228); and promoters of various Agrobacterium genes (e.g., U.S. Pat. Nos. 4,771,002; 5,102,796; 5,182,200; and 5,428,147).

Suitable inducible promoters for use in plants include the promoter from the ACE1 system which responds to copper (Mett et al., Proc. Natl. Acad. Sci. USA, 90:4567-4571 (1993)); the promoter of the maize In2 gene which responds to benzenesulfonamide herbicide safeners (Hershey et al., Mol. Gen. Genetics, 227:229-237 (1991); and Gatz et al., Mol. Gen. Genetics, 243:32-38 (1994)); and the promoter of the Tet repressor from Tn10 (Gatz et al., Mol. Gen. Genet., 227:229-237 (1991)). Another inducible promoter for use in plants is one that responds to an inducing agent to which plants do not normally respond. An exemplary inducible promoter of this type is the inducible promoter from a steroid hormone gene, the transcriptional activity of which is induced by a glucocorticosteroid hormone (Schena et al., Proc. Natl. Acad. Sci. USA, 88:10421 (1991)) or the recent application of a chimeric transcription activator, XVE, for use in an estrogen receptor-based inducible plant expression system activated by estradiol (Zuo et al., Plant J., 24:265-273 (2000)). Other inducible promoters for use in plants are described in EP 332104, PCT International Publication Nos. WO 93/21334 and WO 97/06269. Promoters composed of portions of other promoters and partially or totally synthetic promoters can also be used (see e.g., Ni et al., Plant J., 7:661-676 (1995) and PCT International Publication No. WO 95/14098 describing such promoters for use in plants).

Tissue-specific or tissue-preferential promoters useful for the expression of the novel dense and erect panicle genes of the invention in plants, particularly maize, are those that direct expression in root, pith, leaf or pollen. Such promoters are disclosed in WO 93/07278. Other tissue specific promoters useful in the present invention include the cotton rubisco promoter disclosed in U.S. Pat. No. 6,040,504; the rice sucrose synthase promoter disclosed in U.S. Pat. No. 5,604,121; and the cestrum yellow leaf curling virus promoter disclosed in PCT International Publication No. WO 01/73087. Chemically inducible promoters useful for directing the expression of the novel dense and erect panicle gene in plants are disclosed in U.S. Pat. No. 5,614,395.

The promoter may include, or be modified to include, one or more enhancer elements to thereby provide for higher levels of transcription. Suitable enhancer elements for use in plants include the PClSV enhancer element (U.S. Pat. No. 5,850,019), the CaMV 35S enhancer element (U.S. Pat. Nos. 5,106,739 and 5,164,316) and the FMV enhancer element (Maiti et al., Transgenic Res., 6:143-156 (1997)). See also PCT International Publication No. WO 96/23898.

Such constructs can contain a ‘signal sequence’ or ‘leader sequence’ to facilitate co-translational or post-translational transport of the peptide of interest to certain intracellular structures such as the chloroplast (or other plastid), endoplasmic reticulum, or Golgi apparatus, or to be secreted. For example, the construct can be engineered to contain a signal peptide to facilitate transfer of the peptide to the endoplasmic reticulum. A signal sequence is known or suspected to result in cotranslational or post-translational peptide transport across the cell membrane. In eukaryotes, this typically involves secretion into the Golgi apparatus, with some resulting glycosylation. A leader sequence refers to any sequence that, when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids including chloroplasts, mitochondria, and the like. Plant expression cassettes may also contain an intron, such that mRNA processing of the intron is required for expression.

Such constructs can also contain 5′ and 3′ untranslated regions. A 3′ untranslated region is a polynucleotide located downstream of a coding sequence. Polyadenylation signal sequences and other sequences encoding regulatory signals capable of affecting the addition of polyadenylic acid tracts to the 3′ end of the mRNA precursor are 3′ untranslated regions. A 5′ untranslated region is a polynucleotide located upstream of a coding sequence.

The termination region may be native with the transcriptional initiation region, may be native with the sequence of the present invention, or may be derived from another source. Convenient termination regions are available from the Ti-plasmid of A. tumefaciens, such as the octopine synthase and nopaline synthase termination regions (see e.g., Guerineau et al., Mol. Gen. Genet., 262:141-144 (1991); Proudfoot, Cell, 64:671-674 (1991); Sanfacon et al., Genes Dev., 5:141-149 (1991); Mogen et al., Plant Cell, 2:1261-1272 (1990); Munroe et al., Gene, 91:151-158 (1990); Ballas et al., Nucleic Acids Res., 17:7891-7903 (1989); and Joshi et al., Nucleic Acid Res., 15:9627-9639 (1987)).

Where appropriate, the vector and DEP1 sequences may be optimized for increased expression in the transformed host cell. That is, the sequences can be synthesized using host cell-preferred codons for improving expression, or may be synthesized using codons at a host-preferred codon usage frequency. Generally, the GC content of the polynucleotide will be increased (see e.g., Campbell et al., Plant Physiol., 92:1-11 (1990) for a discussion of host-preferred codon usage). Methods are known in the art for synthesizing host-preferred polynucleotides (see e.g., U.S. Pat. Nos. 6,320,100; 6,075,185; 5,380,831; and 5,436,391, U.S. Application Publication Nos. 20040005600 and 20010003849, and Murray et al., Nucleic Acids Res., 17:477-498 (1989).

In certain embodiments, polynucleotides of interest are targeted to the chloroplast for expression. In this manner, where the polynucleotide of interest is not directly inserted into the chloroplast, the expression cassette may additionally contain a polynucleotide encoding a transit peptide to direct the nucleotide of interest to the chloroplasts. Such transit peptides are known in the art (see e.g., Von Heijne et al., Plant Mol. Biol. Rep., 9:104-126 (1991); Clark et al., J. Biol. Chem., 264:17544-17550 (1989); Della-Cioppa et al., Plant Physiol., 84:965-968 (1987); Romer et al., Biochem. Biophys. Res. Commun., 196:1414-1421 (1993); and Shah et al., Science, 233:478-481 (1986)). The polynucleotides of interest to be targeted to the chloroplast may be optimized for expression in the chloroplast to account for differences in codon usage between the plant nucleus and this organelle. In this manner, the polynucleotides of interest may be synthesized using chloroplast-preferred codons (see e.g., U.S. Pat. No. 5,380,831).

A plant expression cassette (i.e., a DEP1 open reading frame operatively linked to a promoter) can be inserted into a plant transformation vector, which allows for the transformation of DNA into a cell. Such a molecule may consist of one or more expression cassettes, and may be organized into more than one vector DNA molecule. For example, binary vectors are plant transformation vectors that utilize two non-contiguous DNA vectors to encode all requisite cis- and trans-acting functions for transformation of plant cells (Hellens et al., Trends in Plant Science, 5:446-451 (2000)).

A plant transformation vector comprises one or more DNA vectors for achieving plant transformation. For example, it is a common practice in the art to utilize plant transformation vectors that comprise more than one contiguous DNA segment. These vectors are often referred to in the art as binary vectors. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a polynucleotide of interest (i.e., a polynucleotide engineered to be capable of expression in a plant cell for which generation of transgenic plants is desired).

For certain target species, different antibiotic or herbicide selectable markers may be preferred. Selection markers used routinely in transformation include the nptII gene, which confers resistance to kanamycin and related antibiotics (Messing & Vierra, Gene, 19:259-268 (1982); and Bevan et al., Nature, 304:184-187 (1983)), the bar gene, which confers resistance to the herbicide phosphinothricin (White et al., Nucl. Acids Res., 18: 1062 (1990), and Spencer et al., Theor. Appl. Genet., 79: 625-631 (1990)), the hph gene, which confers resistance to the antibiotic hygromycin (Blochinger & Diggelmann, Mol. Cell. Biol., 4:2929-2931 (1984)), the dhfr gene, which confers resistance to methotrexate (Bourouis et al., EMBO J., 2(7):1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Also present on this plasmid vector are sequences required for bacterial replication. The cis-acting sequences are arranged in a fashion to allow efficient transfer into plant cells and expression therein. For example, the selectable marker sequence and the sequence of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as in understood in the art (Hellens et al., 2000). Several types of Agrobacterium strains (e.g., LBA4404, GV3101, EHA101, EHA105, etc.) can be used for plant transformation. The second plasmid vector is not necessary for introduction of polynucleotides into plants by other methods such as, e.g., microprojection, microinjection, electroporation, and polyethylene glycol.

In another embodiment, a nucleotide sequence of the present invention is directly transformed into a plastid genome. A major advantage of plastid transformation is that plastids are generally capable of expressing bacterial genes without substantial modification, and plastids are capable of expressing multiple open reading frames under control of a single promoter. Plastid transformation technology is extensively described in U.S. Pat. Nos. 5,451,513, 5,545,817 and 5,545,818, in PCT International Application Publication WO 95/16783, and in McBride et al., Proc. Natl. Acad. Sci. USA, 91:7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rpsl2 genes conferring resistance to spectinomycin and/or streptomycin are utilized as selectable markers for transformation (Svab et al., Proc. Natl. Acad. Sci. USA, 87:8526-8530 (1990); Staub et al., Plant Cell, 4:39-45 (1992)). This results in stable homoplasmic transformants at a frequency of approximately one per 100 bombardments of target leaves. The presence of cloning sites between these markers allows creation of a plastid targeting vector for introduction of foreign genes (Staub et al., EMBO J., 12:601-606 (1993)). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., Proc. Natl. Acad. Sci. USA, 90:913-917 (1993)). Previously, this marker had been used successfully for high-frequency transformation of the plastid genome of the green alga Chlamydomonas reinhardtii (Goldschmidt-Clermont, Nucl. Acids Res., 19:4083-4089 (1991)). Other selectable markers useful for plastid transformation are known in the art. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleotide sequence of the present invention is inserted into a plastid-targeting vector and transformed into the plastid genome of a desired plant host. Plants homoplastic for plastid genomes containing a nucleotide sequence of the present invention are obtained, and are preferentially capable of high expression of the nucleotide sequence.

### Host Cells

Host cells are cells into which a heterologous nucleic acid molecule of the invention may be introduced. Representative eukaryotic host cells include yeast and plant cells, as well as prokaryotic hosts such as E. coli and Bacillus subtilis. Preferred host cells for functional assays substantially or completely lack endogenous expression of a DEP1 protein.

A host cell strain may be chosen which modulates the expression of the recombinant sequence, or modifies and processes the gene product in a specific manner. For example, different host cells have characteristic and specific mechanisms for the translational and post-translational processing and modification (e.g., glycosylation, phosphorylation of proteins). Appropriate cell lines or host cells may be chosen to ensure the desired modification and processing of the foreign protein expressed. For example, expression in a bacterial system may be used to produce a non-glycosylated core protein product, and expression in yeast will produce a glycosylated product.

The present invention further encompasses recombinant expression of a DEP1 protein in a stable cell line. Methods for generating a stable cell line following transformation of a heterologous construct into a host cell are known in the art (see e.g., Joyner, Gene Targeting: A Practical Approach, 1993, Oxford University Press, Oxford/New York). Thus, transformed cells, tissues, and plants are understood to encompass not only the end product of a transformation process, but also transgenic progeny or propagated forms thereof.

### DEP1 Knockout Plants

The present invention also provides DEP1 knockout plants comprising a disruption of a DEP1 locus. A disrupted gene may result in expression of an altered level of full-length DEP1 protein or expression of a mutated variant DEP1 protein. Plants with complete or partial functional inactivation of the DEP1 gene may be generated, e.g., by expressing a mutant DEP1 allele (e.g., SEQ ID NO: 1) in the plant.

A knockout plant in accordance with the present invention may also be prepared using anti-sense, double-stranded RNA, or ribozyme DEP1 constructs, driven by a universal or tissue-specific promoter to reduce levels of DEP1 gene expression in somatic cells, thus achieving a “knock-down” phenotype. The present invention also provides the generation of plants with conditional or inducible inactivation of DEP1.

The present invention also encompasses transgenic plants with specific “knocked-in” modifications in the disclosed DEP1 gene, for example to create an over-expression mutant having a dominant negative phenotype. Thus, “knocked-in” modifications include the expression of mutant alleles of the DEP1 gene.

DEP1 knockout plants may be prepared in mocot or dicot plants, such as maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees. Rice, wheat, barley, oat, soybean and rye are particularly contemplated. As used herein, a plant refers to a whole plant, a plant organ (e.g., root, stem, leaf, flower bud, or embryo), a seed, a plant cell, a propagule, an embryo, other plant parts (e.g., protoplasts, pollen, pollen tubes, ovules, embryo sacs, zygotes) and progeny of the same. Plant cells can be differentiated or undifferentiated (e.g., callus, suspension culture cells, protoplasts, leaf cells, root cells, phloem cells, pollen).

For preparation of a DEP1 knockout plant, introduction of a polynucleotide into plant cells is accomplished by one of several techniques known in the art, including but not limited to electroporation or chemical transformation (see e.g., Ausubel, ed. (1994) Current Protocols in Molecular Biology, John Wiley and Sons, Inc., Indianapolis, Ind.). Markers conferring resistance to toxic substances are useful in identifying transformed cells (having taken up and expressed the test polynucleotide sequence) from non-transformed cells (those not containing or not expressing the test polynucleotide sequence). In one aspect of the invention, genes are useful as a marker to assess introduction of DNA into plant cells. Transgenic plants, transformed plants, or stably transformed plants, or cells, tissues or seed of any of the foregoing, refer to plants that have incorporated or integrated exogenous polynucleotides into the plant cell. Stable transformation refers to introduction of a polynucleotide construct into a plant such that it integrates into the genome of the plant and is capable of being inherited by progeny thereof.

In general, plant transformation methods involve transferring heterologous DNA into target plant cells (e.g., immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent (i.e., temperature and/or herbicide). The shoots are then transferred to a selective rooting medium for recovering rooted shoot or plantlet. The transgenic plantlet then grow into mature plant and produce fertile seeds (see e.g., Hiei et al., Plant J., 6:271-282 (1994); and Ishida et al., Nat. Biotechnol., 14:745-750 (1996)). A general description of the techniques and methods for generating transgenic plants are found in Ayres et al., CRC Crit. Rev. Plant Sci., 13:219-239 (1994); and Bommineni et al., Maydica, 42:107-120 (1997). Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Then molecular and biochemical methods can be used for confirming the presence of the integrated nucleotide(s) of interest in the genome of transgenic plant.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods to transfer DNA (see e.g., Hiei et al., Plant J., 6:271-282 (1994); Ishida et al., Nat. Biotechnol., 14:745-750 (1996); Ayres et al., CRC Crit. Rev. Plant Sci., 13:219-239 (1994); and Bommineni et al., Maydica, 1997, 42:107-120 (1997)).

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., Plant Molec. Biol, 8:291-298 (1987)). Alternatively, transformation efficiency may be enhanced by wounding the target tissue to be transformed. Wounding of plant tissue may be achieved, for example, by punching, maceration, bombardment with microprojectiles (see e.g., Bidney et al., Plant Molec. Biol., 18:301-313 (1992).

In one embodiment, the plant cells are transfected with vectors via particle bombardment (i.e., with a gene gun). Particle mediated gene transfer methods are known in the art, are commercially available, and include, but are not limited to, the gas driven gene delivery instrument described in U.S. Pat. No. 5,584,807. This method involves coating the polynucleotide sequence of interest onto heavy metal particles, and accelerating the coated particles under the pressure of compressed gas for delivery to the target tissue.

Other particle bombardment methods are also available for the introduction of heterologous polynucleotide sequences into plant cells. Generally, these methods involve depositing the polynucleotide sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles that maximizes the number of cells receiving particles under uniform conditions, resulting in the introduction of the polynucleotide sample into the target tissue.

Specific initiation signals may also be used to achieve more efficient translation of sequences encoding the polypeptide of interest. Such signals include the ATG initiation codon and adjacent sequences. In cases where sequences encoding the polypeptide of interest, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system that is used, such as those described in the literature (Scharf et al., Results Probl. Cell Differ., 20:125 (1994)).

The cells that have been transformed may be grown into plants in accordance with conventional ways (see e.g., McCormick et al., Plant Cell Rep., 5:81-84 (1986)). These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as transgenic seed) having a polynucleotide of the invention, for example, an expression cassette of the invention, stably incorporated into their genome.

Transgenic plants of the invention can be homozygous for the added polynucleotides; i.e., a transgenic plant that contains two added sequences, one sequence at the same locus on each chromosome of a chromosome pair. A homozygous transgenic plant can be obtained by sexually mating (selfing) an independent segregant transgenic plant that contains the added sequences according to the invention, germinating some of the seed produced and analyzing the resulting plants produced for enhanced enzyme activity (i.e., herbicide resistance) and/or increased plant yield relative to a control (native, non-transgenic) or an independent segregant transgenic plant.

It is to be understood that two different transgenic plants can also be mated to produce offspring that contain two independently segregating added, exogenous polynucleotides. Selfing of appropriate progeny can produce plants that are homozygous for all added, exogenous polynucleotides that encode a polypeptide of the present invention. Back-crossing to a parental plant and outcrossing with a non-transgenic plant are also contemplated.

Following introduction of DNA into plant cells, the transformation or integration of the polynucleotide into the plant genome is confirmed by various methods such as analysis of polynucleotides, polypeptides and metabolites associated with the integrated sequence.

### DEP1 Inhibitors

The present invention further discloses assays to identify DEP1 binding partners and DEP1 inhibitors. DEP1 antagonists/inhibitors are agents that alter chemical and biological activities or properties of a DEP1 protein. Methods of identifying inhibitors involve assaying a reduced level or quality of DEP1 function in the presence of one or more agents. Exemplary DEP1 inhibitors include small molecules as well as biological inhibitors as described herein below.

As used herein, the term “agent” refers to any substance that potentially interacts with a DEP1 nucleic acid or protein, including any of synthetic, recombinant, or natural origin. An agent suspected to interact with a protein may be evaluated for such an interaction using the methods disclosed herein.

Exemplary agents include but are not limited to peptides, proteins, nucleic acids, small molecules (e.g., chemical compounds), antibodies or fragments thereof, nucleic acid-protein fusions, any other affinity agent, and combinations thereof. An agent to be tested may be a purified molecule, a homogenous sample, or a mixture of molecules or compounds.

A small molecule refers to a compound, for example an organic compound, with a molecular weight of less than about 1,000 daltons, more preferably less than about 750 daltons, still more preferably less than about 600 daltons, and still more preferably less than about 500 daltons. A small molecule also preferably has a computed log octanol-water partition coefficient in the range of about −4 to about +14, more preferably in the range of about −2 to about +7.5.

Exemplary nucleic acids that may be used to disrupt DEP1 function include antisense RNA and small interfering RNAs (siRNAs) (see e.g., U.S. Application Publication No. 20060095987. These inhibitory molecules may be prepared based upon the DEP1 gene sequence and known features of inhibitory nucleic acids (see e.g., Van der Krol et al., Plant Cell, 2:291-299 (1990); Napoli et al., Plant Cell, 2:279-289 (1990); English et al., Plant Cell, 8:179-188 (1996); and Waterhouse et al., Nature Rev. Genet., 2003, 4:29-38 (2003).

Agents may be obtained or prepared as a library or collection of molecules. A library may contain a few or a large number of different molecules, varying from about ten molecules to several billion molecules or more. A molecule may comprise a naturally occurring molecule, a recombinant molecule, or a synthetic molecule. A plurality of agents in a library may be assayed simultaneously. Optionally, agents derived from different libraries may be pooled for simultaneous evaluation.

Representative libraries include but are not limited to a peptide library (U.S. Pat. Nos. 6,156,511, 6,107,059, 5,922,545, and 5,223,409), an oligomer library (U.S. Pat. Nos. 5,650,489 and 5,858,670), an aptamer library (U.S. Pat. Nos. 7,338,762; 7,329,742; 6,949,379; 6,180,348; and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that may potentially bind to a DEP1 protein.

A library may comprise a random collection of molecules. Alternatively, a library may comprise a collection of molecules having a bias for a particular sequence, structure, or conformation, for example, as for inhibitory nucleic acids (see e.g., U.S. Pat. Nos. 5,264,563 and 5,824,483). Methods for preparing libraries containing diverse populations of various types of molecules are known in the art, for example as described in U.S. patents cited herein above. Numerous libraries are also commercially available.

A control level or quality of DEP1 activity refers to a level or quality of wild type DEP1 activity, for example, when using a recombinant expression system comprising expression of SEQ ID NO: 2. When evaluating the inhibiting capacity of an agent, a control level or quality of DEP1 activity comprises a level or quality of activity in the absence of the agent. A control level may also be established by a phenotype or other measurable trait.

Methods of identifying DEP1 inhibitors also require that the inhibiting capacity of an agent be assayed. Assaying the inhibiting capacity of an agent may comprise determining a level of DEP1 gene expression; determining DNA binding activity of a recombinantly expressed DEP1 protein; determining an active conformation of a DEP1 protein; or determining a change in a trait in response to binding of a DEP1 inhibitor (e.g., yield, lodging resistance, panicle number, grain number per panicle, dwarf or semi-dwarf stature, photosynthetic efficiency, population growth rate during grain filling period, water transport capacity, mechanical strength of the stem, and dry matter production). In particular embodiments, a method of identifying a DEP1 inhibitor may comprise (a) providing a cell, plant, or plant part expressing a DEP1 protein; (b) contacting the cell, plant, or plant part with an agent; (c) examining the cell, plant, or plant part for a change in a trait as compared to a control; and (d) selecting an agent that induces a change in the trait as compared to a control. Any of the agents so identified in the disclosed inhibitory or binding assays (see hereinafter) may be subsequently applied to a cell, plant or plant part as desired to effectuate a change in that cell, plant or plant part. For example, disruption of a DEP1 gene (e.g., SEQ ID NO: 2) or inhibition of a DEP1 polynucleotide or polypeptide (e.g., SEQ ID NO: 10) would alter one or more plant traits in a desirable way (e.g., increase grain yield).

The present invention also encompasses a rapid and high throughput screening method that relies on the methods described herein. This screening method comprises separately contacting a DEP1 protein with a plurality of agents. In such a screening method the plurality of agents may comprise more than about 10⁴ samples, or more than about 10⁵ samples, or more than about 10⁶ samples.

The in vitro and cellular assays of the invention may comprise soluble assays, or may further comprise a solid phase substrate for immobilizing one or more components of the assay. For example, a DEP1 protein, or a cell expressing a DEP1 protein, may be bound directly to a solid state component via a covalent or non-covalent linkage. Optionally, the binding may include a linker molecule or tag that mediates indirect binding of a DEP1 protein to a substrate.

### DEP1 Binding Assays

The present invention also encompasses methods of identifying of a DEP1 inhibitor by determining specific binding of a substance (e.g., an agent described previously) to a DEP1 protein. For example, a method of identifying a DEP1 binding partner may comprise: (a) providing a DEP1 protein of SEQ ID NO: 2; (b) contacting the DEP1 protein with one or more agents under conditions sufficient for binding; (c) assaying binding of the agent to the isolated DEP1 protein; and (d) selecting an agent that demonstrates specific binding to the DEP1 protein. Specific binding may also encompass a quality or state of mutual action such that binding of an agent to a DEP1 protein is inhibitory.

Specific binding refers to a binding reaction which is determinative of the presence of the protein in a heterogeneous population of proteins and other biological materials. The binding of an agent to a DEP1 protein may be considered specific if the binding affinity is about 1×10⁴ M⁻¹ to about 1×10⁶ M⁻¹ or greater. Specific binding also refers to saturable binding. To demonstrate saturable binding of an agent to a DEP1 protein, Scatchard analysis may be carried out as described, for example, by Mak et al., J. Biol. Chem., 264:21613-21618 (1989).

Several techniques may be used to detect interactions between a DEP1 protein and an agent without employing a known competitive inhibitor. Representative methods include, but are not limited to, Fluorescence Correlation Spectroscopy, Surface-Enhanced Laser Desorption/Ionization Time-Of-flight Spectroscopy, and BIACORE® technology, each technique described herein below. These methods are amenable to automated, high-throughput screening.

Fluorescence Correlation Spectroscopy (FCS) measures the average diffusion rate of a fluorescent molecule within a small sample volume. The sample size may be as low as 10³ fluorescent molecules and the sample volume as low as the cytoplasm of a single bacterium. The diffusion rate is a function of the mass of the molecule and decreases as the mass increases. FCS may therefore be applied to protein-ligand interaction analysis by measuring the change in mass and therefore in diffusion rate of a molecule upon binding. In a typical experiment, the target to be analyzed (e.g., a DEP1 protein) is expressed as a recombinant protein with a sequence tag, such as a poly-histidine sequence, inserted at the N-terminus or C-terminus. The expression is mediated in a host cell, such as E. coli, yeast, Xenopus oocytes, or mammalian cells. The protein is purified using chromatographic methods. For example, the poly-histidine tag may be used to bind the expressed protein to a metal chelate column such as Ni²⁺ chelated on iminodiacetic acid agarose. The protein is then labeled with a fluorescent tag such as carboxytetramethylrhodamine or BODIPY™ reagent (available from Molecular Probes of Eugene, Oreg.). The protein is then exposed in solution to the potential ligand, and its diffusion rate is determined by FCS using instrumentation available from Carl Zeiss, Inc. (Thornwood of New York, N.Y.). Ligand binding is determined by changes in the diffusion rate of the protein.

Surface-Enhanced Laser Desorption/Ionization (SELDI) was developed by Hutchens & Yip, Rapid Commun. Mass Spectrom., 1993, 7:576-580. When coupled to a time-of-flight mass spectrometer (TOF), SELDI provides a technique to rapidly analyze molecules retained on a chip. It may be applied to ligand-protein interaction analysis by covalently binding the target protein, or portion thereof, on the chip and analyzing by mass spectrometry the small molecules that bind to this protein (Worrall et al., Anal Chem., 1998, 70(4):750-756 (1998)). In a typical experiment, a target protein (e.g., a DEP1 protein) is recombinantly expressed and purified. The target protein is bound to a SELDI chip either by utilizing a poly-histidine tag or by other interaction such as ion exchange or hydrophobic interaction. A chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind a target protein are identified by the stringency of the wash needed to elute them.

BIACORE® relies on changes in the refractive index at the surface layer upon binding of a ligand to a target protein (e.g., a DEP1 protein) immobilized on the layer. In this system, a collection of small ligands is injected sequentially in a 2-5 microliter cell, wherein the target protein is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein. In a typical experiment, a target protein is recombinantly expressed, purified, and bound to a BIACORE® chip. Binding may be facilitated by utilizing a poly-histidine tag or by other interaction such as ion exchange or hydrophobic interaction. A chip thus prepared is then exposed to one or more potential ligands via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction (see also Homola et al., Sensors and Actuators, 54:3-15 (1999) and references therein).

### Conformational Assays

The present invention also encompasses methods of identifying DEP1 binding partners and inhibitors that rely on a conformational change of a DEP1 protein when bound by or otherwise interacting with a substance (e.g., an agent described previously). For example, application of circular dichroism to solutions of macromolecules reveals the conformational states of these macromolecules. The technique may distinguish random coil, alpha helix, and beta chain conformational states.

To identify inhibitors of a DEP1 protein, circular dichroism analysis may be performed using a recombinantly expressed DEP1 protein. A DEP1 protein is purified, for example by ion exchange and size exclusion chromatography, and mixed with an agent. The mixture is subjected to circular dichroism. The conformation of a DEP1 protein in the presence of an agent is compared to a conformation of a DEP1 protein in the absence of the agent. A change in conformational state of a DEP1 protein in the presence of an agent identifies a DEP1 binding partner or inhibitor. Representative methods are described in U.S. Pat. Nos. 5,776,859 and 5,780,242. Antagonistic activity of the inhibitor may be assessed using functional assays, such assaying nitrate content, nitrate uptake, lateral root growth, or plant biomass, as described herein.

In accordance with the disclosed methods, cells expressing DEP1 may be provided in the form of a kit useful for performing an assay of DEP1 function. For example, a kit for detecting a DEP1 may include cells transfected with DNA encoding a full-length DEP1 protein and a medium for growing the cells.

Assays of DEP1 activity that employ transiently transfected cells may include a marker that distinguishes transfected cells from non-transfected cells. A marker may be encoded by or otherwise associated with a construct for DEP1 expression, such that cells are simultaneously transfected with a nucleic acid molecule encoding DEP1 and the marker. Representative detectable molecules that are useful as markers include but are not limited to a heterologous nucleic acid, a protein encoded by a transfected construct (e.g., an enzyme or a fluorescent protein), a binding protein, and an antigen.

Assays employing cells expressing recombinant DEP1 or plants expressing DEP1 may additionally employ control cells or plants that are substantially devoid of native DEP1 and, optionally, proteins substantially similar to a DEP1 protein. When using transiently transfected cells, a control cell may comprise, for example, an untransfected host cell. When using a stable cell line expressing a DEP1 protein, a control cell may comprise, for example, a parent cell line used to derive the DEP1-expressing cell line.

### Anti-DEP1 Antibodies

In another aspect of the invention, a method is provided for producing an antibody that specifically binds a DEP1 protein. According to the method, a full-length recombinant DEP1 protein is formulated so that it may be used as an effective immunogen, and used to immunize an animal so as to generate an immune response in the animal. The immune response is characterized by the production of antibodies that may be collected from the blood serum of the animal.

An antibody is an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Antibodies of the invention include diabodies, tetrameric antibodies, single chain antibodies, tetravalent antibodies, multispecific antibodies (e.g., bispecific antibodies), and domain-specific antibodies that recognize a particular epitope. Cell lines that produce anti-DEP1 antibodies are also encompassed by the invention.

Specific binding of an antibody to a DEP1 protein refers to preferential binding to a DEP1 protein in a heterogeneous sample comprising multiple different antigens. Substantially lacking binding describes binding of an antibody to a control protein or sample, i.e., a level of binding characterized as non-specific or background binding. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher.

DEP1 antibodies prepared as disclosed herein may be used in methods known in the art relating to the expression and activity of DEP1 proteins, e.g., for cloning of nucleic acids encoding a DEP1 protein, immunopurification of a DEP1 protein, and detecting a DEP1 protein in a plant sample, and measuring levels of a DEP1 protein in plant samples. To perform such methods, an antibody of the present invention may further comprise a detectable label, including but not limited to a radioactive label, a fluorescent label, an epitope label, and a label that may be detected in vivo. Methods for selection of a label suitable for a particular detection technique, and methods for conjugating to or otherwise associating a detectable label with an antibody are known to one skilled in the art.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Comparison Between Rice Varieties Shao 313 and Shao 314

Shao 314 and the precursor to Shao 313 have a background of “Wuyunjing” and were found in the field of Shaoxing Institute of Agricultural Science, Zhejiang Province. Shao 313 was obtained from its precursor by multiple generations of backcross and selection using Shao 314 as recurrent parent, in which 8 generations of backcross were completed.

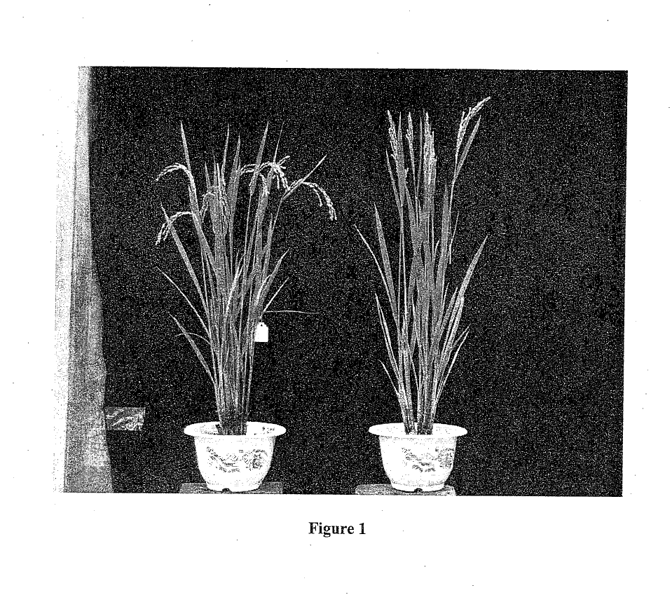

Lines Shao 313 and Shao 314 are near-isogenic, though Shao 313 has a dense and erect panicle (see the right side of FIG. 1) and Shao 314 has curved and loose panicle (see the left side of FIG. 1). Sample seeds of both lines were deposited in the China General Microbiological Culture Collection Center (CGMCC, address: Institute of Microbiology, Chinese Academy of Sciences, Beijing, China, P.O. Box 2714, postal code: 100080) on 8 May 2008 under accession numbers of CGMCC No. 2485 and CGMCC No. 2486, respectively. The above deposit was converted into a deposit under the Budapest Treaty on the International Recognition of the Deposit of Microorganisms for the Purposes of Patent Procedure on 13 May 2008.

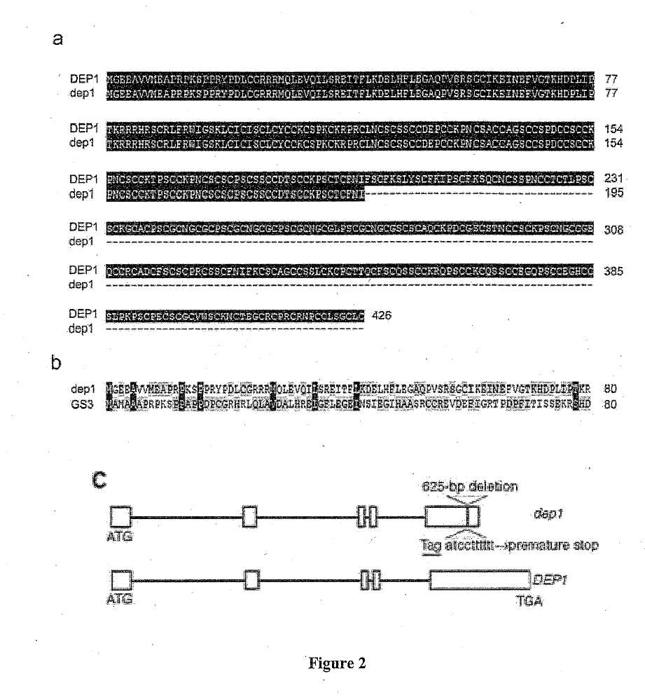

Each of these two lines express different alleles of the dense and erect panicle gene. Shao 314 expresses the DEP1 allele, and the resultant protein DEP1 is a phosphatidylethanolamine-binding protein-like domain protein, which shares some homology with the N terminus of GS3 (FIG. 2b).

Shao 313 expresses an allele (dep1) that acts as a dominant negative regulator of panicle architecture and grain number. dep1 differs from DEP1 in that in dep1 a 637-bp stretch of the middle of exon 5 is replaced by a 12-bp sequence, which has the effect of creating a premature stop codon and consequently the loss of 230 residues from the C terminus of the resultant protein (FIGS. 2a, c).

Seeds from Shao 313 and Shao 314 were sowed in the test field of Changping Farm in Beijing in June 2007, and 30 panicles from main tillers of each plant were obtained after the plants had grown to maturity. The primary branch number (pb), secondary branch number (sb), panicle length (PL) and number of grains on each panicle were counted. The measurement of 1000-grain weight was performed by splitting purified grains using a sample splitter or quarter method to obtain a sample having a weight near the specified weight, precisely weighing the sample to obtain its actual weight (W), then counting the grains to obtain grain number (m), and calculating the 1000-grain weight by using the following formula: 1000-grain weight (g/1000 grains)=W/m×1000. A test result was allowed if the difference between tests is less than 0.4 g for a 1000-grain weight of less than 20 g, 0.7 g for a 1000-grain weight of between 20.1 g and 50 g, and 1.0 g for a 1000-grain weight of greater than 50.1 g.

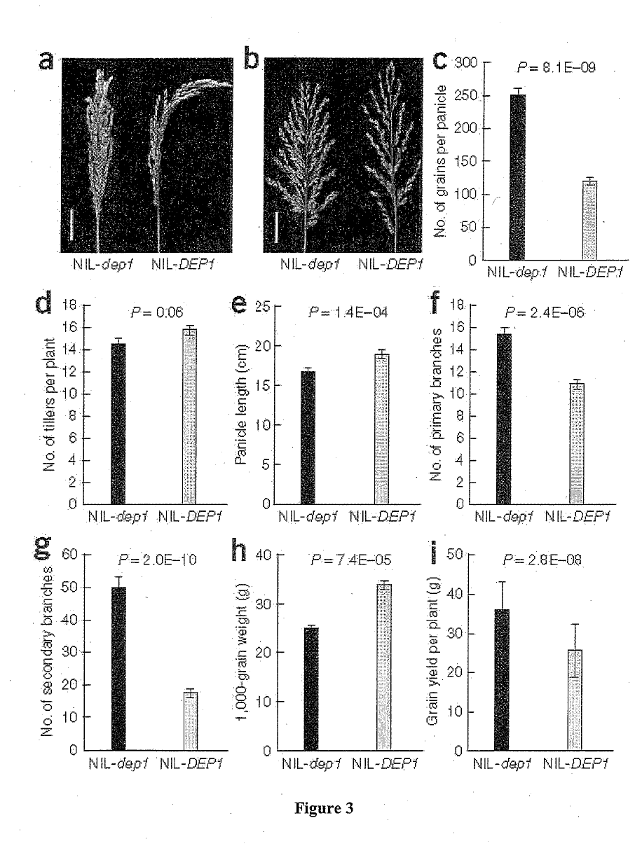

As shown in FIG. 3, the allelic constitution at the DEP1 locus affects grain yield. In a field comparison, the lines did not differ from one another with respect to either the heading date, the length of the grain-filling period or culm number (FIG. 3d). However, grain number per main panicle was significantly higher in the presence of dep1 (FIG. 3c), and there were clear differences in panicle architecture, inflorescence internode and panicle length (FIGS. 3b, e), and the number of both primary (FIGS. 3b, f) and secondary (FIG. 3g) branches per panicle. Increasing the number of grains can be associated with incomplete grain filling, but there was no evidence for grain-filling failure in the presence of dep1. The 1,000-grain weight of NIL-dep1 plants was slightly less than that of NIL-DEP1 plants (FIG. 3h), but the overall grain yield per plant under field conditions was increased (+40.9%) (FIG. 3i).

### Example 2

### Measurement of Photosynthesis and Chlorophyll Content

The measurement of chlorophyll content was performed on rice during the four-leaf stage by collecting leaves of corresponding sites of 313 and 314 respectively, weighing them, and testing chlorophyll content by an ethanol method (Shen, Comm. Phytophysiology, 3:62-64 (1988)). Light absorption peaks at 665 nm and 649 nm were determined, and chlorophyll content was calculated using the following formula: chlorophyll content (mg/g)=pigment concentration (c)×volume of extract liquid×dilution factor/fresh or dry weight of sample. Photosynthesis efficiencies of 313 and 314 were measured between 9:00 AM and 10:00 AM by a method comprising: using photosynthesis system L1-6400 (LI-COR Inc., Lincoln, Nebr. USA), setting different light intensities (250, 500, 750, 1000, 1500, 2000, 2500 μmol photons m⁻² sec⁻¹), and measuring net absorptions of CO₂ (μmol m⁻² sec⁻¹) under corresponding light intensities. Each test was repeated twice.

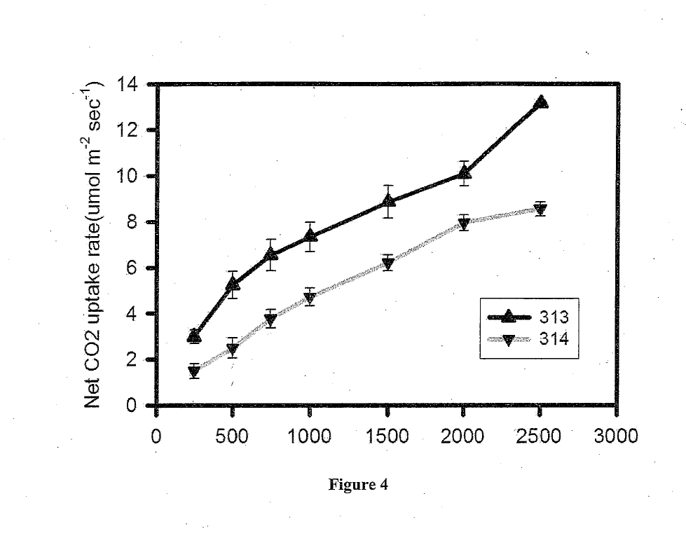

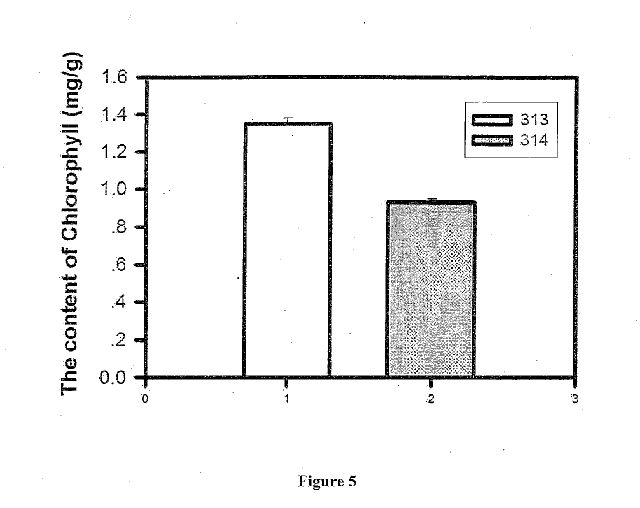

As shown in FIGS. 4 and 5, Shao 313 had significantly higher photosynthesis efficiency and significantly higher chlorophyll content than Shao 314. The results showed that the erect panicle rice variety has better light energy utilization, which would lead to higher photosynthesis efficiency and higher yield. This indicates that the dense and erect panicle rice variety has the potential to increase yield as compared to the curved and loose panicle rice variety.

### Example 3

### Measurement of Vascular Bundle Number

Immature uppermost internodes from top and flag leaves of 313 and 314 were collected and fixed for more than 48 hours by using FAA fixing solution. They were subsequently dehydrated for 30 minutes by using sequentially 40%, 60%, 80%, 95% and 95% anhydrous ethanol. Internodes were subsequently washed with 100% anhydrous ethanol and historesin (Leica Historesin embedding kit, lot 010066, 2022 18500) in a ratio of 3:1 for 3-4 hours, 100% anhydrous ethanol and historesin in a ratio of 1:1 for 3-4 hours, 100% anhydrous ethanol and historesin in a ratio of 1:3 for 3-4 hours, washing twice with 100% historesin, in which the second washing was sustained overnight, and washing with fresh historesin for 1 hour in the next morning. Washed internodes were embedded using 100% historesin and hardener (Leica Historesin embedding kit, lot 010066, 2022 18500) in a ratio of 16:1, and sealed with parafilm. After the embedding agent was sufficiently solidified (for 1-2 days), samples were sliced to a thickness of 8-10 μm, dyed (e.g., blue dye), and observed under microscope.

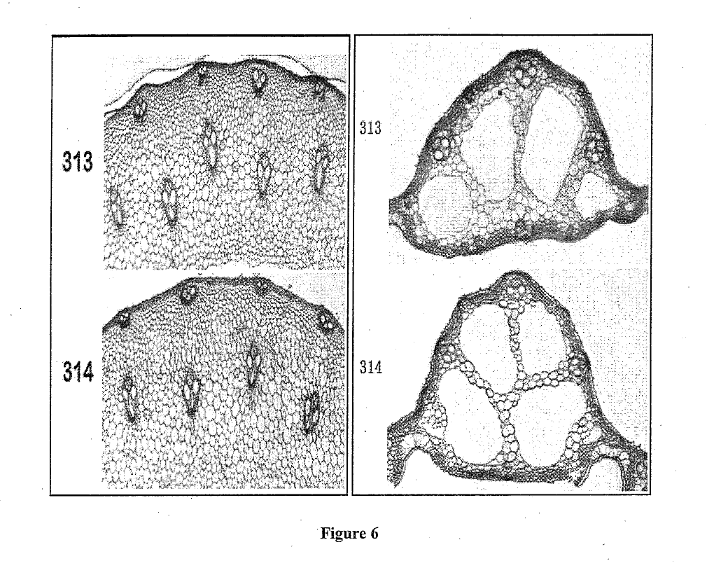

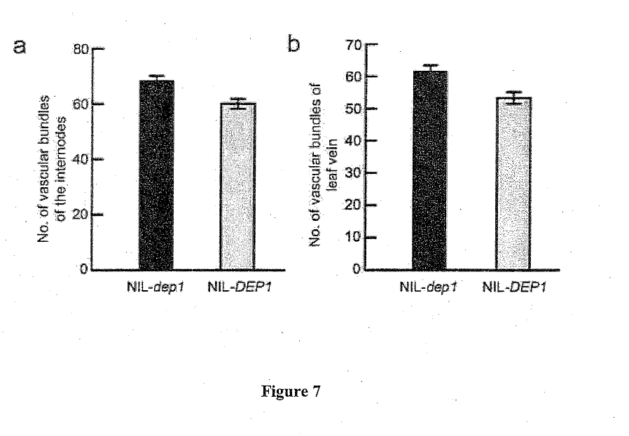

As shown in FIGS. 6 and 7, the transverse resin slices of stem of uppermost internode from top showed that large and small vascular bundles of rice stem were arranged in two concentric circles. Shao 313 had 34 large vascular bundles and 34 small vascular bundles, while Shao 314 had 30 large vascular bundles and 30 small vascular bundles (see FIG. 6, left side). Thus, 313 had 68 vascular bundles in the stem of uppermost internode from top, while 314 had 60, which is to say that the erect panicle rice variety had 8 more vascular bundles than the curved panicle rice variety (see FIG. 7, left side). The transverse slices of flag leaves showed that 313 had 10 midribs (see FIG. 6, upper right) while 314 had 8 midribs (see FIG. 6, lower right).

Based upon theses observations, one would conclude that the vascular system of NIL-dep1 plants was better developed and their sclerenchyma cell walls were thicker at maturity than those in NIL-DEP1 plants. These traits are favorable for both water transport capacity and the mechanical strength of the stem, both of which are important factors for the breeding of high-yielding, lodging-resistant varieties of plants such as rice.

### Example 4

### Cloning and Sequencing Rice Dep1 Gene, Obtaining Genomic Sequence, and Isolating Promoter and 3′UTR Region

By using F2 segregation population and employing a map-based cloning method, a Balilla type dense and erect panicle gene, dep1, was cloned. The promoter region of the gene was also isolated. Specifically, to clone the dep1 gene several populations were constructed. A major QTL (quantitative trait loci) in charge of dense and erect panicle trait was firstly localized by using northeast dense and erect panicle variety “Shennong 265” and “Qianchonglang” in combination with japonica rice varieties Nipponbare and “Zhonghua 11” respectively. The QTL was located on the long arm of chromosome 9 between two SSR markers, RM3700 and RM7424.

In order to precisely localize dep1, a larger F2 population was constructed. The japonica rice variety W101 comprising the dep1 gene was hybridized to NJ6, and another japonica rice variety Q169 comprising the dep1 gene was hybridized to 93-11. The F2 population was obtained after self cross of F1, and 1600 individual plants exhibiting curved panicle were chosen therefrom. Using these 1600 individual plants, the dep1 gene was localized on BAC AP005419 in a region of 85 Kb between the newly developed STS markers S2 (5′-cttcaactgcctgcgagaccacc-3′ (SEQ ID NO: 15) and 5′-gcttgactgacataatgccgcta-3′ (SEQ ID NO: 16)) and S11-2 (5′-taagccgatgattactccagac-3′ (SEQ ID NO: 17) and 5′-gttcatttaaagaagtcctcaccg-3′ (SEQ ID NO: 18)), a region comprising 14 possible genes. These 14 genes were sequences and compared, but only one of these genes was different between two parent plants, and was provisionally named dep1 gene. dep1 and DEP1 full length cDNAs were separately amplified by using primers dep1-F: 5′-gctctagagtcgactcaacataagcaaccactgaga-3′ (SEQ ID NO: 19) and dep1-R: 5′-gctctagagtcgacctagatgttgaagcaggtgcag-3′ (SEQ ID NO: 20), and using the cDNA of 313 and 314 as templates. A promoter sequence of 1.9 Kb was amplified by using primers 5′-cggaattcgtctctcagtgagccgttcc-3′ (SEQ ID NO: 21) and 5′-cgggatcctcatgggcattatagcagca-3′ (SEQ ID NO: 22) and using the genomic DNA of 313 as a template.

As a result, the following sequences were identified: a dep1 cDNA sequence obtained from 313 (SEQ ID NO: 1); a DEP1 cDNA sequence obtained from 314 (SEQ ID NO: 2); the DEP1 gDNA (genomic DNA) sequence obtained from 314 (SEQ ID NO: 3); the dep1 promoter sequence obtained from 313 (SEQ ID NO: 4); the dep1 protein sequence obtained from 313 (SEQ ID NO: 9); and the DEP1 protein sequence (obtained from 314) (SEQ ID NO: 10).

### Example 5

### Complementation Study

A complementary vector was created by first isolating the promoter and 3′UTR region of the dep1 gene, and then inserting the ORF of dep1 between them, and finally inserting the combined sequence into a pCAMBI1300 vector to construct pdep::dep1, which was transferred into Agrobacterium GV3101 and then transferred into 314 by an agrobacterium mediated method.

Specifically, the 900 bp 3′UTR region of the dep1 gene was amplified by using primers 5′-ctgcagtcgtaacccatgctgtctca-3′ (SEQ ID NO: 23) and 5′-aagctttggcgagtaaatgagtccaa-3′ (SEQ ID NO: 24), which contain the restriction enzyme cleavage sites for Pst I and Hind III, respectively, using genomic DNA of Shao 313 (NIL-dep1, comprising the near-isogenic line of dep1 gene) as a template, and then was inserted into a pBLUESCRIPT® vector (Stratagene, La Jolla, Calif.). After verification by sequencing, the recombinant vector was cleaved by Pst I and Hind III, and the cleaved fragment was linked to the binary vector pCAMBI1300 to create pCAMBI1300-3′UTR. The 2 Kb promoter sequence of the dept gene was amplified by using primers 5′-gaattcgtctctcagtgagccgttcc-3′ (SEQ ID NO: 25) and 5′-ggatcctcatgggcattatagcagca-3′ (SEQ ID NO: 26), which contain the restriction enzyme cleavage sites for EcoR I and BamH I, respectively. Using the genomic DNA of Shaoxing 313 as a template, the fragment was inserted into a pBLUESCRIPT® vector (Stratagene, La Jolla, Calif.). After verification by sequencing, the recombinant vector was cleaved via EcoR I and BamH I, and the fragment was linked to the pCAMBI1300-3′UTR plasmid cleaved by the same enzymes to construct pCAMBI1300-DEPP:3′UTR. The 588 bp cDNA sequence of the dep1 gene was amplified by using primers 5′-cgggatccatgggggaggaggcggtggtgatg-3′ (SEQ ID NO: 27) and 5′-gtcgactcaacataagcaaccactgaga-3′ (SEQ ID NO: 28), which contain the restriction enzyme cleavage sites for BamH I and Sal I, respectively, and using the cDNA of Shaoxing 313 as a template. The obtained fragment was inserted into a pGEM® 18T vector (Takala). After verification by sequencing, the recombinant vector was subject to digestion of both BamH I and Sal I, the obtained fragment was linked to the pCAMBI1300-DEPP:3′UTR plasmid and cleaved by the same two enzymes to construct complementary vector pCAMBI1300-DEPP:dep1-3′UTR. The constructed vector was transferred into Agrobacterium AGL1 and then transferred into Shaoxing 314 via an agrobacterium-mediated method as follows.

Rice seeds from which glume were removed were placed in a triangular flask, sterilized using 70% alcohol for 3 minutes, then sterilized using a 2.5% NaClO (sodium hypochlorite) solution for 45 minutes. The sterilized seeds were washed with sterilized water several times under aseptic conditions, and transferred to NB induction media (N6 media macroelements, B5 media microelements, vitamin B₅, iron salt, enzymatic casein hydrolysate 300 mg/L, proline 500 mg/L, sucrose 30 g/L, inositol 100 mg/L, pH 5.8), and cultured at 26° C. under darkness with the embryo placed upward.

Calluses were obtained after culturing for about one month. The desirable callus, which was dry, dispersed and white-yellow, was selected and placed on a fresh induction culture medium and then subcultured once for two weeks. Calluses were subsequently transformed through a co-culture method mediated by agrobacterium (see Hiei et al., Plant J., 6(2):271-282 (1994)). Agrobacterium AGL1 was cultured one day in advance. Agrobacterial broth in the logarithmic growth phase was collected and centrifuged for 15 minutes at 3,000 rpm. The bacterium was re-suspended in 20 ml of N₆B₅G I+AS transformation culture media (N6 media macroelements, B5 media microelements, vitamin B₅, iron salt, sucrose 40 g/L, glucose 20 g/L, pH 5.2; and 100 mmol/L acetosyringone (AS)), and the suspension was diluted until the OD₆₀₀ equaled about 0.5. The desirable callus was picked and placed in the suspension and co-cultured with the agrobacterium for 30 minutes (optionally with shaking by a shaker). Then the callus was removed and directly placed on a solid NB+AS co-culturing media (N6 media macroelements, B5 media microelements, vitamin B₅, iron salt, enzymatic casein hydrolysate 300 mg/L, proline 500 mg/L, sucrose 30 g/L, inositol 100 mg/L, pH 5.8, and 100 mmol/L acetosyringone (AS)), and cultured at 26° C. under darkness.

The transformed callus was collected after co-culturing for 3 days, washed three times with sterilized water supplemented with 500 mg/L carbenicillin, and then washed once with N₆B₅G II liquid culture media (N6 media macroelements, B5 media microelements, vitamin B₅, iron salt, sucrose 20 g/L, glucose 10 g/L, pH 5.8) supplemented with 500 mg/L carbenicillin. The callus was placed on a layer of filter paper in a sterilized culture dish to absorb agrobacterium liquid on the surface of callus and subsequently placed in a selection culture media (N6 media macroelements, B5 media microelements, vitamin B₅, iron salt, enzymatic casein hydrolysate 300 mg/L, proline 500 mg/L, sucrose 30 g/L, inositol 100 mg/L, pH 5.8, 500 mg/L cefotaxime, 50 mg/L hygromycin B) and cultured at 26° C. under darkness.

After selection, the normally growing callus was picked and placed into a differentiation culture medium (MS media macroelements, MS media microelements, MS vitamins, iron salt, sucrose 30 g/L, tryptophan 50 mg/L, NAA 0.1 mg/L, GELRITE® (curing agent, Beijing Zhentai Company) 2.6 g/L, pH 5.8) and cultured at 26° C. under lighting conditions suitable for differentiation. Optionally, expanding propagation could be conducted in a callus induction NB culture medium for preventing contamination. Usually, shoots could be differentiated after about one month.

The differentiated shoots were selected and subsequently moved to a rooting media (½ MS, ½ B5 organo, sucrose 10 g/L, GELRITE® (curing agent) 2.6 g/L, pH 5.8), and rooted under suitable lighting conditions for about one month to obtain a substantially normal plantlet which could be transplanted to soil. Plantlets were transplanted so as to preserve moisture.

In addition, a pDEP1:RNAi-DEP1 construct was also created using methods similar to those described above, and the construct was transferred into Shao 313 plants using an agrobacterium-mediated method as described above. The pDEP1:RNAi-DEP1 construct was based on the sequence of a 300-bp fragment of the N terminus of the DEP1 cDNA sequence, which shows no significant homology with any other sequences in the rice genome.

The ingredients of each of the media described in this Example is provided as follows: N6 macroelements (per 1000 mL)—KNO₃ 56.6 g, (NH₄)₂SO₄ 9.26 g, MgSO₄.7H₂O 3.70 g, KH₂PO₄ 8.00 g, CaCl₂.2H₂O 3.32 g; N6 microelements (per 500 mL) MnSO₄.H₂O 165 mg, MnSO₄.4H₂O 220 mg, ZnSO₄.7H₂O 75 mg, H₃BO₃ 80 mg, KI 40 mg; B5 microelements (per 500 mL) MnSO₄.4H₂O 500 mg, H₃BO₃ 150 mg, ZnSO₄.7H₂O 10 mg, KI 37.5 mg, NaMoO₄.2H₂O 12.5 mg, CuSO₄.5H₂O 1.25 mg CoCl₂.6H₂O 1.25 mg; B5-organo (per 500 mL) V_{B1} 500 mg, V_{B6} 50 mg, nicotinic acid 50 mg; Fe-Salt (per 500 mL) FeSO₄.7H₂O 1.39 g, Na-EDTA 1.87 g; N6 vitamin (per 500 mL) nicotinic acid 25 mg, thiamine hydrochloride (V_{B1}) 5 mg, pyridoxine hydrochloride (V_{B6}) 5 mg, glycine (aminoacetic acid) 100 mg, inositol 5 g; MS macroelements (per 1000 mL) KNO₃ 38 g, NH₄NO₃ 33 g, KH₂PO₄ 3.4 g, MgSO₄.7H₂O 7.4 g, CaCl₂.2H₂O 8.8 g; MS microelements (per 500 mL) MnSO₄.4H₂O 1115 mg, ZnSO₄.7H₂O 430 mg, H₃BO₃ 310 mg, KI 41.5 mg, NaMoO₄.2H₂O 2.5 mg, CuSO₄.5H₂O 1.25 mg, CoCl₂.6H₂O 1.25 mg; MS vitamin (per 500 mL) glycine (aminoacetic acid) 100 mg, thiamine hydrochloride (V_{B1}) 20 mg, pyridoxine hydrochloride (V_{B6}) 25 mg, nicotinic acid 25 mg, inositol 5 g.

As would be appreciated by one of ordinary skill in the art, the principle of a complementation test is to introduce a dominant gene into a receptor without the gene, in which if the phenotype of the receptor plant becomes the phenotype of that exhibited by the introduced gene, it indicates that the gene is the one controlling the phenotype.

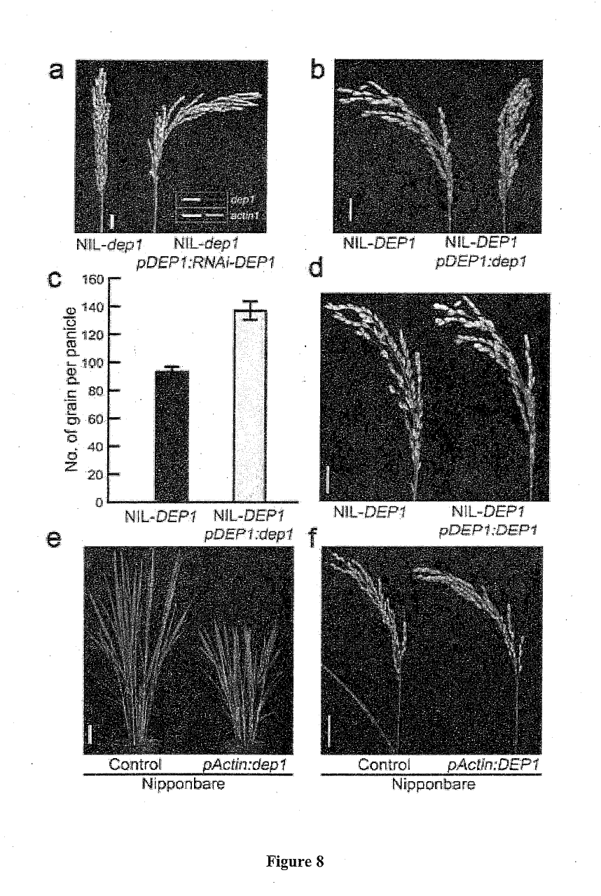

As shown in FIG. 8, transgenic Shao 313 (NIL-dep1) individuals carrying a pDEP1:RNAi-DEP1 construct had curved panicles, elongated inflorescence internodes and fewer grains per panicle (FIG. 8a). Transgenic Shao 314 (NIL-DEP1) plants expressing mutant DEP1 allele (dep1) under the control of its native promoter had a semi-dwarf stature, but had the same erect panicle as Shao 313 plants, along with an increased number of grains per panicle, shorter inflorescence internodes, and an increased number of both primary and secondary panicle branches (FIGS. 8b, c). In contrast, transgenic Shao 314 plants carrying a pDEP1:DEP1 construct showed no noticeable change in panicle architecture (FIG. 8d). All the transgenic Nipponbare plants, in which dep1 was constitutively expressed under the control of a rice actin1 promoter, were severely dwarfed (FIG. 8e) with erect panicles, whereas Actin:DEP1 plants were unchanged with respect to either panicle size or plant architecture (FIG. 8f). These observations reinforce the conclusion that dep1 acts as a dominant negative regulator of panicle architecture and grain number. Furthermore, since Shao 314 lacks the dep1 gene, and Shao 314 exhibited the 313-like phenotype after introduction of dep1, it indicates that the cloned dep1 gene is responsible for this phenotype and can be used to transform plants such as rice into a desirable phenotype.

### Example 6

### Overexpression Study

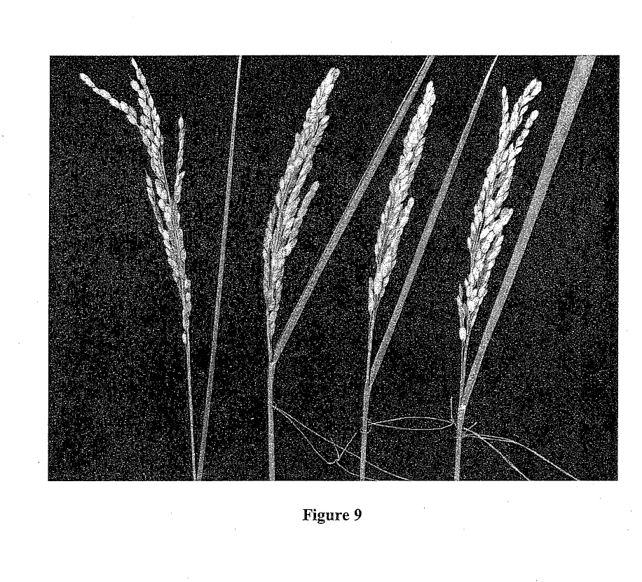

An overexpression vector was created by inserting the ORF of the dep1 gene into a pCAMBI-2300-Actin construct resulting in pAct::dep1. Specifically, the 588 bp cDNA sequence of the dep1 gene was amplified by using primers 5′-cgggatccatgggggaggaggcggtggtgatg-3′ (SEQ ID NO: 27) and 5′-gtcgactcaacataagcaaccactgaga-3′ (SEQ ID NO: 28), which are restriction enzyme cleavage sites for BamH I and Sal I, respectively, using the cDNA of 313 as a template. The cleaved fragment was subsequently inserted to a pGEM 18T vector (Takala). After verification by sequencing, the recombinant vector was subject to digestion of both BamH I and Sal I, and the obtained fragment was linked to a pCAMBI-2300-Actin plasmid cleaved by the same two enzymes to construct overexpression vector pAct::dep1. The overexpression vector was transferred into Agrobacterium AGL1, and then transferred into japonica rice Nipponbare via an agrobacterium-mediated method similar to that described previously. As demonstrated in FIG. 9, overexpression of the dep1 gene resulted in panicles becoming more dense and erect.

### Example 7

### Effect of Dep1 on Grain Yield

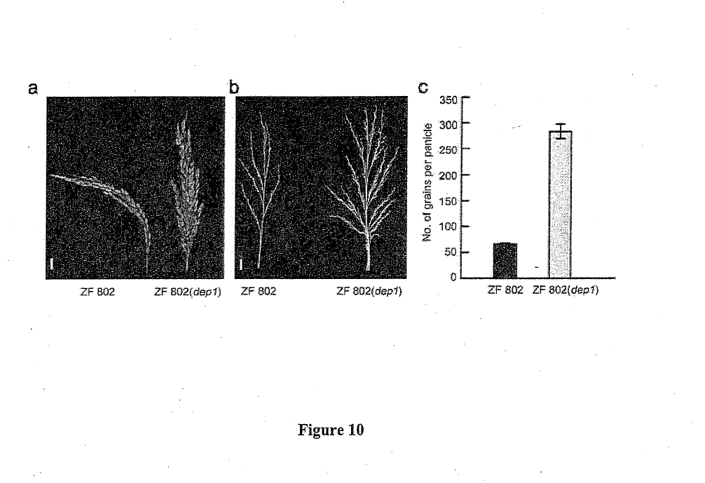

The effect of dep1 on grain yield was tested in an indica background by backcrossing the dep1 segment present in the japonica variety Wuyunjing 7 into the indica variety Zhefu 802. As shown in FIG. 10, the NIL, ZF 802 (dep1), produced more grains per panicle and out-yielded its recurrent parent. This is further evidence that dep1 is a useful allele for increasing grain yield in plants such as rice.

### Example 8

### Transcription Level Study

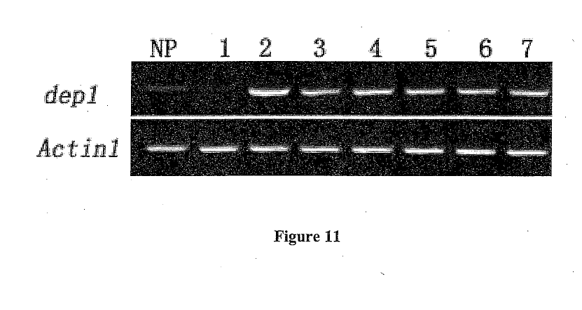

Total RNAs were extracted from leaves of different transgenic plant lines and cDNAs were obtained by reverse transcription for RT-PCR. The extraction of RNA was conducted by using TRIZOL® (Invitrogen, New Zealand). The cDNA template was prepared in accordance with the instructions of reverse transcriptase (Promega, USA). Internal reference primers were Actin1-F: agcaactgggatgatatgga (SEQ ID NO: 31), and Actin-R: cagggcgatgtaggaaagc (SEQ ID NO: 32), and dep1 gene specific primers were gcgagatcacgttcctcaag (SEQ ID NO: 33) and tgcagtttggcttacagcat (SEQ ID NO: 34). For PCR, a 25 μl reaction system comprised 1 μl cDNA template, 5 nmol forward primer and 5 nmol reverse primer, 2.5 μl 10×PCR buffer (Shenggong, Shanghai), 0 2 mmol/L each dNTP, 1.5 mmol/L MgCl₂, 1 U Taq DNA polymerase (Shenggong, Shanghai), and balanced ddH₂O. The PCR reaction procedure was carried out at 94° C. for 3 minutes, repeating 94° C. for 30 seconds, 60° C. for 45 seconds and 72° C. for 1.5 minutes 28 times, then extending at 72° C. for 10 minutes. The annealing temperature depended on the primers. The PCR product was assayed on 1% agarose gel. As shown in FIG. 11, transcription levels of dep1 in different transgenic Nipponbare plant lines were elevated to different extents as compared to the control non-transgenic Nipponbare.

### Example 9

### Tissue Expression Study

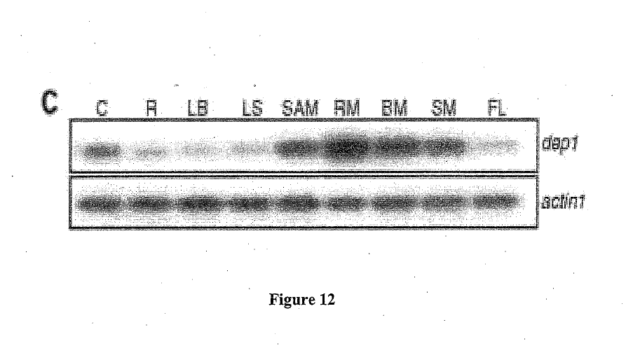

Total RNA was extracted from various parts of Shao 313 plants using TRIZOL®. RNAs were separately reverse transcribed to obtain cDNAs. The cDNAs of each of these tissues were amplified by using dep1 specific primers described previously and assayed by electrophoresis. As shown in FIG. 12, semi-quantitative RT-PCR showed that dep1 was present in the root, leaf, culm, inflorescence meristem and young inflorescence NIL-dep1 plants, with the highest expression in the inflorescence meristem at the stage of primary and secondary rachis branch formation.

### Example 10

### Allelic Variation of the DEP1 Locus in Rice Varieties

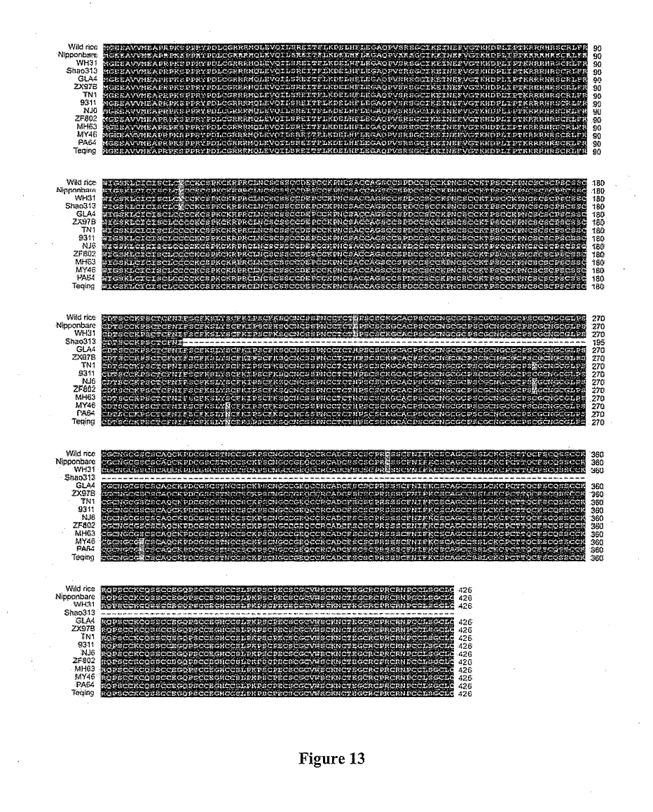

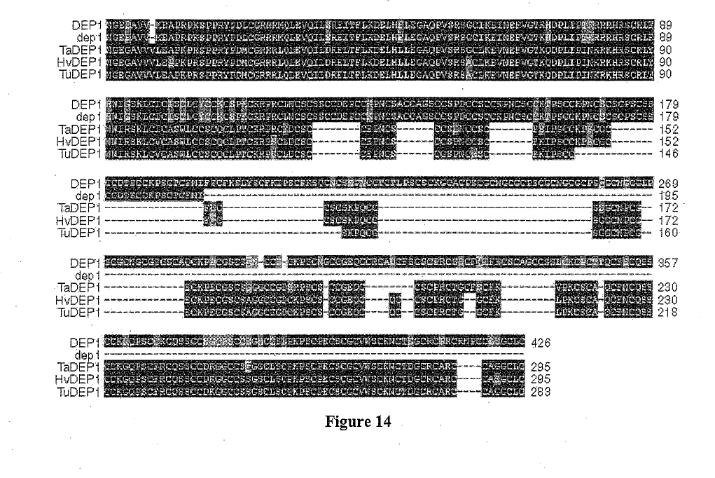

Pedigree records show that many high-yielding Chinese japonica varieties, including Shennong 265, were derived from the Italian land race Balilla, which was extensively cultivated in Italy in the 1970s and introduced into China in 1958. The allelic constitution at the DEP1 locus was explored by re-sequencing from a panel of widely cultivated Chinese varieties (69 japonica and 83 indica). This truncated mutation was present in Balilla and all 36 japonica types having an erect or semi-erect panicle, including super high-yielding cultivars Liaojing 5 and Qianchonglang, but it was absent from all the other varieties. Thus, this natural allelic variation in DEP1 has clearly been exploited by japonica breeding programs in China. Several sequence variants at the DEP1 C terminus were present in the sample of indica types. The variety 93-11 differed from the japonica variety Nipponbare by three amino acids, whereas that of the variety Teqing differed by two amino acids. The Nipponbare sequence differed from that of an accession of Oryza rufipogon by one nucleotide at position 663, but this did not produce a variant peptide (FIG. 13).

### Example 11

### Isolation of Homologous Genes from Other Plants

cDNA sequences homologous to dep1 were identified in wheat, barley and maize by database searches. Homologous EST sequences were searched respectively in EST databases of wheat and barley by performing Basic Logical Alignment Search Tool (BLAST) alignment in the databases provided by NCBI website (www.ncbi.nih.nlm.gov) using the cDNA sequence of rice dep1 as a probe, and these EST sequences were joined from head to tail.

SEQ ID NO: 5 is the cDNA sequence of the dep1 homolog in wheat (TaDEP1), and the corresponding protein sequence is shown in SEQ ID NO: 11. SEQ ID NO: 6 is the dep1 homolog in barley (HvDEP1), and the corresponding protein sequence is shown in SEQ ID NO: 12. SEQ ID NO: 7 is a first dep1 homolog in maize, and the corresponding protein sequence is shown in SEQ ID NO: 13. SEQ ID NO: 8 is a first dep1 homolog in maize, and the corresponding protein sequence is shown in SEQ ID NO: 14. TaDEP1 exhibited 49.1% similarity to OsDEP1 (rice) and 59.3% similarity to Osdep1 (rice). HvDEP1 exhibited 49.1% similarity to OsDEP1 (rice) and 58.3% similarity to Osdep1 (rice).

RNA sequences of wheat and barley were separately extracted and reverse transcribed to obtain cDNA sequences as described previously, primers were designed, and the cDNA sequences of wheat and barley were used respectively as templates for RT-PCR to amplify the ORFs of TaDEP1 and HvDEP1. The primers for amplifying TaDEP1 were 5′-cgggatccatgggggagggcgcggtggt-3′ (SEQ ID NO: 35), and 5′-gcgtcgacttaacacaggcacccgccagca-3′ (SEQ ID NO: 36). The two ends had enzymatic cleavage sites BamH I and Sal I, and the two ends had enzymatic cleavage sites XbaI and SalI. The PCR reaction system comprised a cDNA template 50-100 nmol, 10 μL 5× Phusion Buffer, 200 nmol/L dNTPs, 200 nmol/L up- and down-stream primers, 1 U Phusion enzyme, balanced with ddH₂O to a total volume of 50 μL. The PCR reaction was carried out at 98° C. for 10 seconds, 98° C. for 10 seconds, 60° C. 15 for seconds, 72° C. for 30 seconds, for a total of 35 cycles. The PCR products were assayed in 1% agarose gel and the linked pBLUESCRIPT® SK(+) was recovered. The linked products were used to transform DH 5a competent cells (preserved in the lab) and constructed into a vector pBLUESCRIPT SK− TaDEP1. After confirming by sequencing, TaDEP1 was cleaved with BamH I and Sal I, and the obtained fragments were linked to plasmid pCAMBIA-2300-Actin cleaved by the same enzymes to construct vector pAct::TaDEP1 for overexpression.

The primers for amplifying HvDEP1 were 5′-gctctagaatgggggagggcgcggtggt-3′ (SEQ ID NO: 37) and 5′-acgcgtcgactcaacacaggcacccgctagca-3′ (SEQ ID NO: 38), and the two ends had enzymatic cleavage sites XbaI and SalI. The amplifying method was the same as previously described. The amplified product was linked to pBLUESCRIPT®SK(+) (preserved in the lab) and used to construct vector pBLUESCRIPT® SK-HvDEP1. After verification by sequencing, HvDEP1 was cleaved with Xba I and Sal I, the obtained fragments were linked to plasmid pCAMBIA-2300-Actin cleaved by the same enzymes to construct vector pAct::HyDEP1 for overexpression. The plasmids pAct::TaDEP1 and pAct::HvDEP1 were respectively transformed into Agrobacterium AGL1, and then transformed into Nipponbare via agrobacterium as described previously.

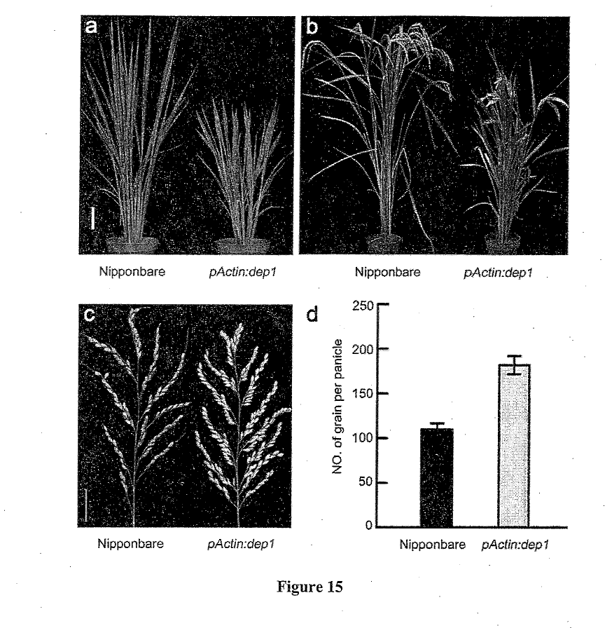

As shown in FIG. 15, the transgenic positive Nipponbare plants exhibited a phenotype similar to that observed with dep1 in rice: lowered plant height, increased numbers of first and second branch of panicles upon maturation, and significantly increased grain number per panicle. This indicates that homologous dept genes from other species (e.g., wheat and barley) have similar functions to those in rice, and may be used in the same manner as described herein for the rice dept gene.

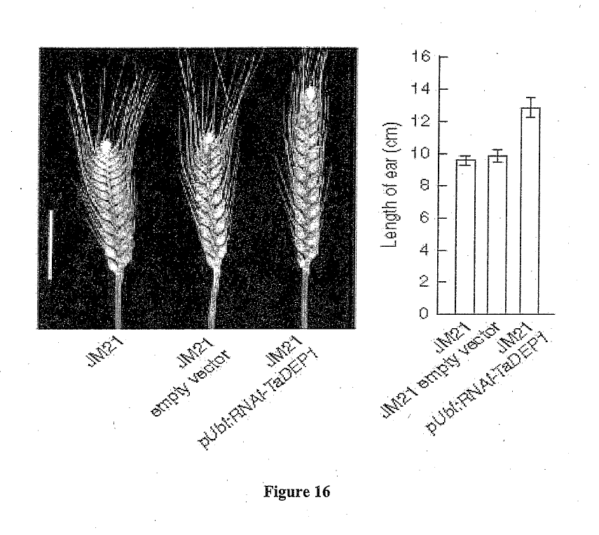

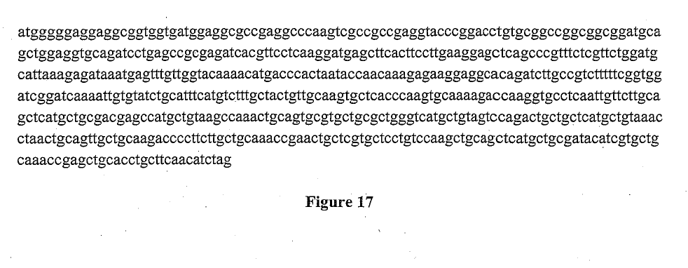

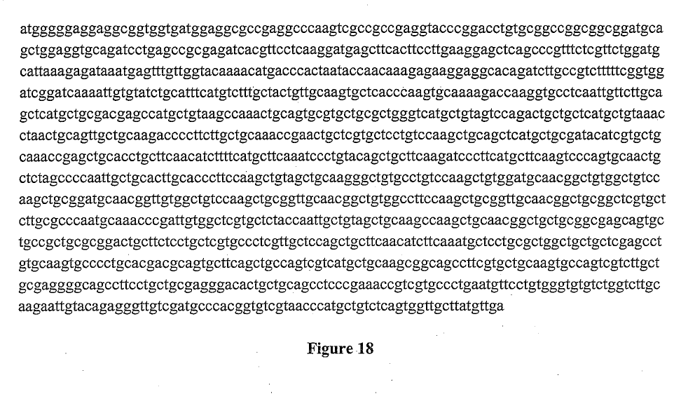

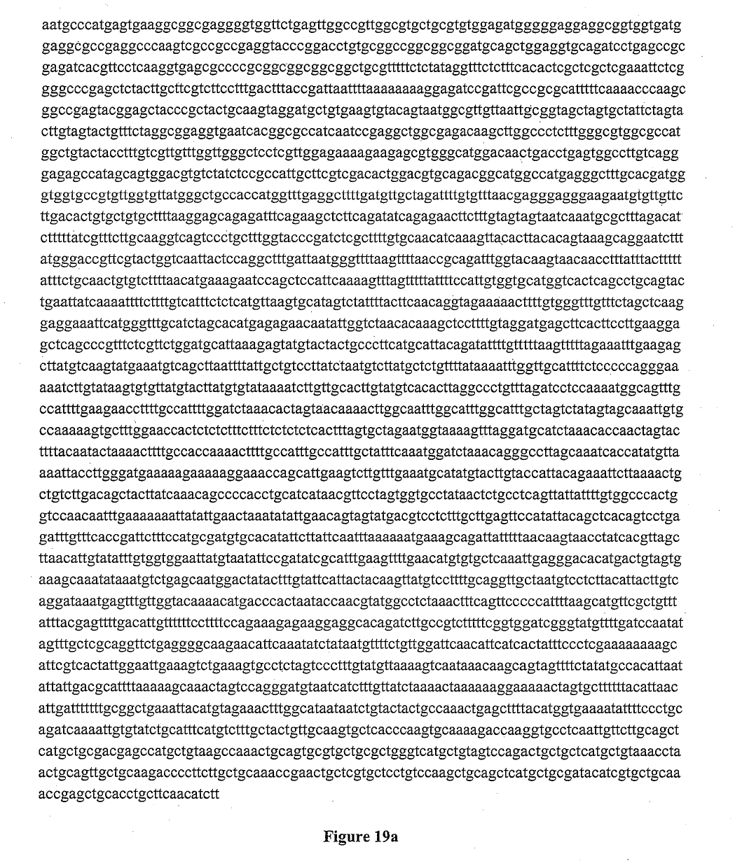

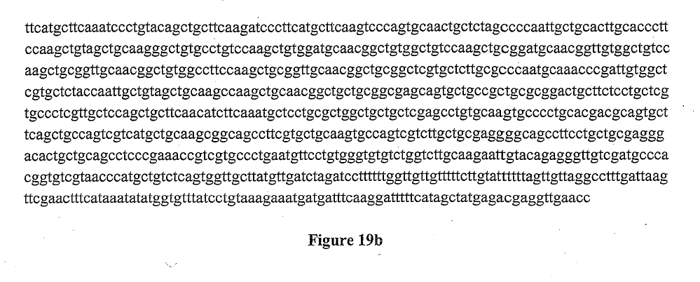

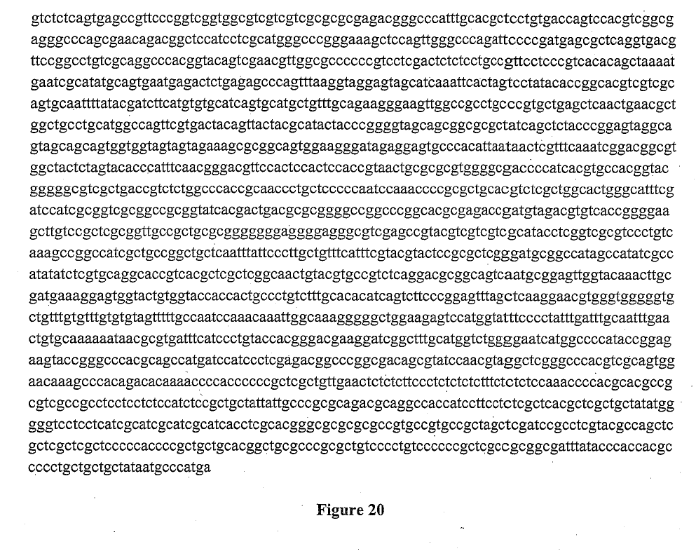

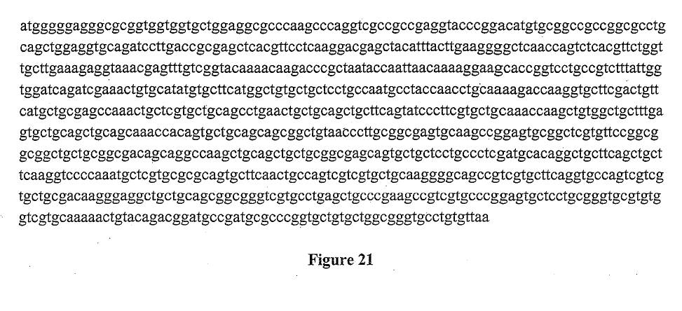

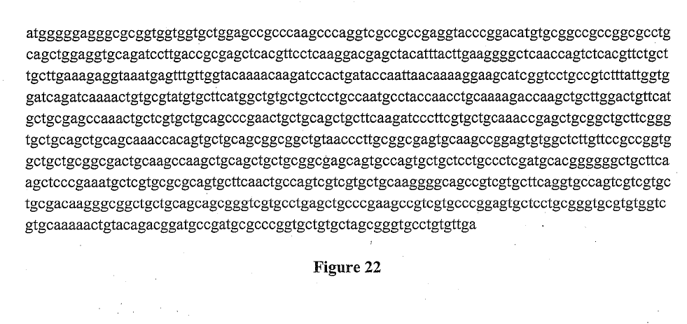

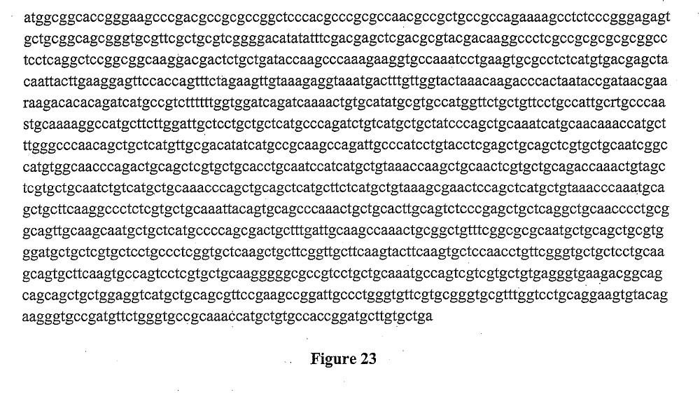

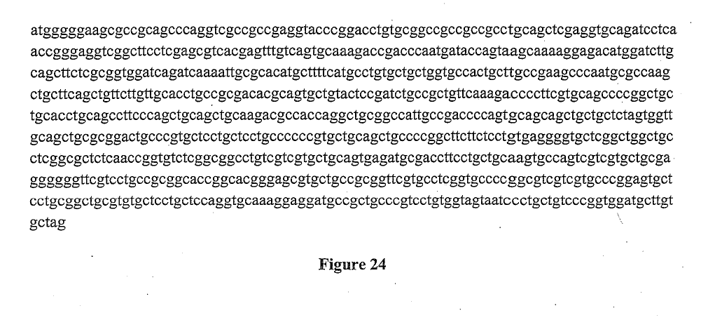

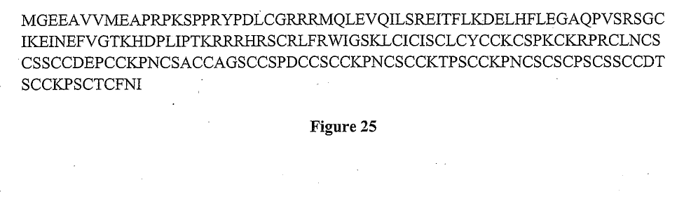

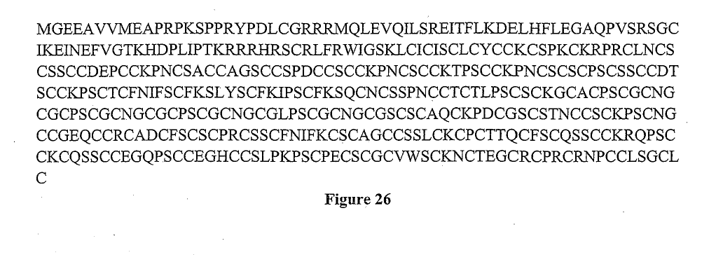

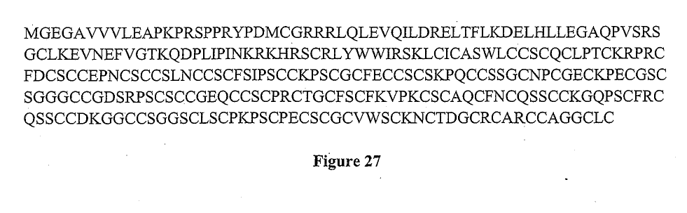

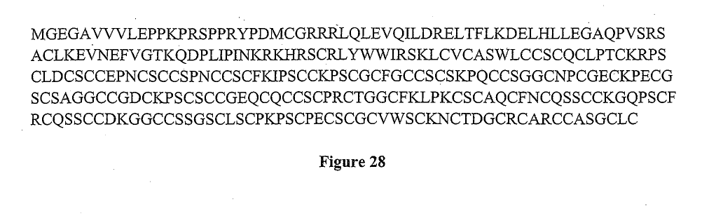

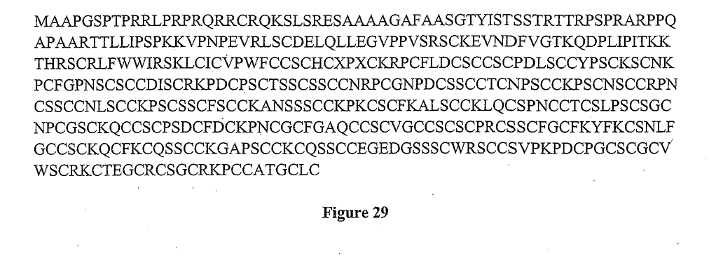

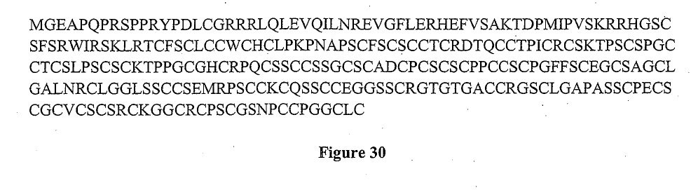

Further, to determine whether any novel gain-of-function was induced by the presence of these truncated genes, number of transgenic wheat plants carrying a pUbi:RNAi-TaDEP1 construct were generated. A 250-bp fragment was amplified from the bread wheat variety Ni982105 to generate the pUbi:RNAi-TaDEP1 construct. As shown in FIG. 16, the consequent downregulation of TaDEP1 resulted in an increase in the length of the ear, a less compact ear and a somewhat reduced number of spikelets.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1. An isolated DEP1 polynucleotide selected from the group consisting of:
(a) a nucleic acid comprising a nucleotide sequence of any one of SEQ ID NOs:1 and 5-8;
(b) a nucleic acid comprising an open reading frame encoding a DEP1 protein comprising a polypeptide sequence of any one of SEQ ID NOs:9 and 11-14; and
(c) a nucleic acid comprising a nucleotide sequence that is the complement of any one of (a)-(c).

2. A vector comprising the isolated DEP1 polynucleotide of claim 1.

3. A host cell which expresses the vector of claim 2.

4. The host cell of claim 3, wherein the cell is selected from the group consisting of animal cell, plant cell, and microorganism cell.

5. A transgenic plant or seed comprising the host cell of claim 4.

6. The transgenic plant or seed of claim 5, wherein the plant is a monocot.

7. The transgenic plant or seed claim 5, wherein the plant is a dicot.

8. The transgenic plant or seed of claim 5, wherein the transgenic plant is selected from the group consisting of maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, and Arabidopsis.

9. An isolated DEP1 polypeptide, comprising
an amino acid sequence of any one of SEQ ID NOs:9 and 11-14.

10. A method for producing a transgenic plant comprising regenerating a transgenic plant from the host cell according to claim 3.

11. A method for producing a transgenic plant comprising crossing a transgenic plant according to claim 5 with a non-transgenic plant.

12. A plant produced by the method according to claim 11 or a transgenic seed derived therefrom.

13. A method of altering a trait in a plant comprising expressing the isolated polynucleotide of claim 1 in the plant.

14. The method of claim 13, wherein the trait is selected from the group consisting of yield, lodging resistance, panicle number, grain number per panicle, dwarf or semi-dwarf stature, photosynthetic efficiency, population growth rate during grain filling period, water transport capacity, mechanical strength of the stem, and dry matter production.

15-29. (canceled)

30. The transgenic plant or seed of claim 5, wherein the transgenic plant is rice.


SEQUENCE LISTING (33 sequences)

SEQ ID NO: 1 - DNA, 588 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggatgagcttcacttccttgaaggagctcagcccgtttctcgttctggatgcattaaagagataaatgagtttgttggtacaaaacatgacccactaataccaacaaagagaaggaggcacagatcttgccgtctttttcggtggatcggatcaaaattgtgtatctgcatttcatgtctttgctactgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatctag

SEQ ID NO: 2 - DNA, 1281 bp, Oryza sativa
atgggggaggaggcggtggtgatggaggcgccgaggcccaagtcgccgccgaggtacccggacctgtgcggccggcggcggatgcagctggaggtgcagatcctgagccgcgagatcacgttcctcaaggatgagcttcacttccttgaaggagctcagcccgtttctcgttctggatgcattaaagagataaatgagtttgttggtacaaaacatgacccactaataccaacaaagagaaggaggcacagatcttgccgtctttttcggtggatcggatcaaaattgtgtatctgcatttcatgtctttgctactgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacagctgcttcaagatcccttcatgcttcaagtcccagtgcaactgctctagccccaattgctgcacttgcacccttccaagctgtagctgcaagggctgtgcctgtccaagctgtggatgcaacggctgtggctgtccaagctgcggatgcaacggttgtggctgtccaagctgcggttgcaacggctgtggccttccaagctgcggttgcaacggctgcggctcgtgctcttgcgcccaatgcaaacccgattgtggctcgtgctctaccaattgctgtagctgcaagccaagctgcaacggctgctgcggcgagcagtgctgccgctgcgcggactgcttctcctgctcgtgccctcgttgctccagctgcttcaacatcttcaaatgctcctgcgctggctgctgctcgagcctgtgcaagtgcccctgcacgacgcagtgcttcagctgccagtcgtcatgctgcaagcggcagccttcgtgctgcaagtgccagtcgtcttgctgcgaggggcagccttcctgctgcgagggacactgctgcagcctcccgaaaccgtcgtgccctgaatgttcctgtgggtgtgtctggtcttgcaagaattgtacagagggttgtcgatgcccacggtgtcgtaacccatgctgtctcagtggttgcttatgttga

SEQ ID NO: 3 - DNA, 4363 bp, Oryza sativa
aatgcccatgagtgaaggcggcgaggggtggttctgagttggccgttggcgtgctgcgtgtggagatgggggaggaggcggtggtgatggaggcgccgaggcccaagtcgccgccgaggtacccggacctgtgcggccggcggcggatgcagctggaggtgcagatcctgagccgcgagatcacgttcctcaaggtgagcgccccgcggcggcggcggctgcgtttttctctataggtttctctttcacactcgctcgctcgaaattctcggggcccgagctctacttgcttcgtcttcctttgactttaccgattaattttaaaaaaaaggagatccgattcgccgcgcatttttcaaaacccaagcggccgagtacggagctacccgctactgcaagtaggatgctgtgaagtgtacagtaatggcgttgttaattgcggtagctagtgctattctagtacttgtagtactgtttctaggcggaggtgaatcacggcgccatcaatccgaggctggcgagacaagcttggccctctttgggcgtggcgccatggctgtactacctttgtcgttgtttggttgggctcctcgttggagaaaagaagagcgtgggcatggacaactgacctgagtggccttgtcagggagagccatagcagtggacgtgtctatctccgccattgcttcgtcgacactggacgtgcagacggcatggccatgagggctttgcacgatgggtggtgccgtgttggtgttatgggctgccaccatggtttgaggcttttgatgttgctagattttgtgtttaacgagggagggaagaatgtgttgttcttgacactgtgctgtgcttttaaggagcagagatttcagaagctcttcagatatcagagaacttctttgtagtagtaatcaaatgcgctttagacatctttttatcgtttcttgcaaggtcagtccctgctttggtacccgatctcgcttttgtgcaacatcaaagttacacttacacagtaaagcaggaatctttatgggaccgttcgtactggtcaattactccaggctttgattaatgggttttaagttttaaccgcagatttggtacaagtaacaacctttatttactttttatttctgcaactgtgtcttttaacatgaaagaatccagctccattcaaaagtttagtttttattttccattgtggtgcatggtcactcagcctgcagtactgaattatcaaaattttcttttgtcatttctctcatgttaagtgcatagtctattttacttcaacaggtagaaaaacttttgtgggtttgtttctagctcaaggaggaaattcatgggtttgcatctagcacatgagagaacaatattggtctaacacaaagctccttttgtaggatgagcttcacttccttgaaggagctcagcccgtttctcgttctggatgcattaaagagtatgtactactgcccttcatgcattacagatattttgtttttaagtttttagaaatttgaagagcttatgtcaagtatgaaatgtcagcttaattttattgctgtccttatctaatgtcttatgctctgttttataaaatttggttgcattttctcccccagggaaaaatcttgtataagtgtgttatgtacttatgtgtataaaatcttgttgcacttgtatgtcacacttaggccctgtttagatcctccaaaatggcagtttgccattttgaagaaccttttgccattttggatctaaacactagtaacaaaacttggcaatttggcatttggcatttgctagtctatagtagcaaattgtgccaaaaagtgctttggaaccactctctctttctttctctctctcactttagtgctagaatggtaaaagtttaggatgcatctaaacaccaactagtacttttacaatactaaaacttttgccaccaaaacttttgccatttgccatttgctatttcaaatggatctaaacagggccttagcaaatcaccatatgttaaaattaccttgggatgaaaaagaaaaaggaaaccagcattgaagtcttgtttgaaatgcatatgtacttgtaccattacagaaattcttaaaactgctgtcttgacagctacttatcaaacagccccacctgcatcataacgttcctagtggtgcctataactctgcctcagttattattttgtggcccactggtccaacaatttgaaaaaaattatattgaactaaatatattgaacagtagtatgacgtcctctttgcttgagttccatattacagctcacagtcctgagatttgtttcaccgattctttccatgcgatgtgcacatattcttattcaatttaaaaaatgaaagcagattatttttaacaagtaacctatcacgttagcttaacattgtatatttgtggtggaattatgtaatattccgatatcgcatttgaagttttgaacatgtgtgctcaaattgagggacacatgactgtagtgaaagcaaatataaatgtctgagcaatggactatactttgtattcattactacaagttatgtccttttgcaggttgctaatgtcctcttacattacttgtcaggataaatgagtttgttggtacaaaacatgacccactaataccaacgtatggcctctaaactttcagttcccccattttaagcatgttcgctgtttatttacgagttttgacattgttttttccttttccagaaagagaaggaggcacagatcttgccgtctttttcggtggatcgggtatgttttgatccaatatagtttgctcgcaggttctgaggggcaagaacattcaaatatctataatgttttctgttggattcaacattcatcactatttccctcgaaaaaaaagcattcgtcactattggaattgaaagtctgaaagtgcctctagtccctttgtatgttaaaagtcaataaacaagcagtagttttctatatgccacattaatattattgacgcattttaaaaagcaaactagtccagggatgtaatcatctttgttatctaaaactaaaaaaggaaaaactagtgcttttttacattaacattgatttttttgcggctgaaattacatgtagaaactttggcataataatctgtactactgccaaactgagcttttacatggtgaaaatattttccctgcagatcaaaattgtgtatctgcatttcatgtctttgctactgttgcaagtgctcacccaagtgcaaaagaccaaggtgcctcaattgttcttgcagctcatgctgcgacgagccatgctgtaagccaaactgcagtgcgtgctgcgctgggtcatgctgtagtccagactgctgctcatgctgtaaacctaactgcagttgctgcaagaccccttcttgctgcaaaccgaactgctcgtgctcctgtccaagctgcagctcatgctgcgatacatcgtgctgcaaaccgagctgcacctgcttcaacatcttttcatgcttcaaatccctgtacagctgcttcaagatcccttcatgcttcaagtcccagtgcaactgctctagccccaattgctgcacttgcacccttccaagctgtagctgcaagggctgtgcctgtccaagctgtggatgcaacggctgtggctgtccaagctgcggatgcaacggttgtggctgtccaagctgcggttgcaacggctgtggccttccaagctgcggttgcaacggctgcggctcgtgctcttgcgcccaatgcaaacccgattgtggctcgtgctctaccaattgctgtagctgcaagccaagctgcaacggctgctgcggcgagcagtgctgccgctgcgcggactgcttctcctgctcgtgccctcgttgctccagctgcttcaacatcttcaaatgctcctgcgctggctgctgctcgagcctgtgcaagtgcccctgcacgacgcagtgcttcagctgccagtcgtcatgctgcaagcggcagccttcgtgctgcaagtgccagtcgtcttgctgcgaggggcagccttcctgctgcgagggacactgctgcagcctcccgaaaccgtcgtgccctgaatgttcctgtgggtgtgtctggtcttgcaagaattgtacagagggttgtcgatgcccacggtgtcgtaacccatgctgtctcagtggttgcttatgttgatctagatccttttttggttgttgtttttcttgtattttttagttgttaggcctttgattaagttcgaactttcataaatatatggtgtttatcctgtaaagaaatgatgatttcaaggatttttcatagctatgagacgaggttgaacc

SEQ ID NO: 4 - DNA, 1959 bp, Oryza sativa
gtctctcagtgagccgttcccggtcggtggcgtcgtcgtcgcgcgcgagacgggcccatttgcacgctcctgtgaccagtccacgtcggcgagggcccagcgaacagacggctccatcctcgcatgggcccgggaaagctccagttgggcccagattccccgatgagcgctcaggtgacgttccggcctgtcgcaggcccacggtacagtcgaacgttggcgccccccgtcctcgactctctcctgccgttcctcccgtcacacagctaaaatgaatcgcatatgcagtgaatgagactctgagagcccagtttaaggtaggagtagcatcaaattcactagtcctatacaccggcacgtcgtcgcagtgcaattttatacgatcttcatgtgtgcatcagtgcatgctgtttgcagaagggaagttggccgcctgcccgtgctgagctcaactgaacgctggctgcctgcatggccagttcgtgactacagttactacgcatactacccggggtagcagcggcgcgctatcagctctacccggagtaggcagtagcagcagtggtggtagtagtagaaagcgcggcagtggaagggatagaggagtgcccacattaataactcgtttcaaatcggacggcgtggctactctagtacacccatttcaacgggacgttccactccactccaccgtaactgcgcgcgtggggcgaccccatcacgtgccacggtacgggggcgtcgctgaccgtctctggcccaccgcaaccctgctcccccaatccaaaccccgcgctgcacgtctcgctggcactgggcatttcgatccatcgcggtcgcggccgcggtatcacgactgacgcgcggggccggcccggcacgcgagaccgatgtagacgtgtcaccggggaagcttgtccgctcgcggttgccgctgcgcgggggggaggggagggcgtcgagccgtacgtcgtcgtcgcatacctcggtcgcgtccctgtcaaagccggccatcgctgccggctgctcaatttattcccttgctgtttcatttcgtacgtactccgcgctcgggatgcggccatagccatatcgccatatatctcgtgcaggcaccgtcacgctcgctcggcaactgtacgtgccgtctcaggacgcggcagtcaatgcggagttggtacaaacttgcgatgaaaggagtggtactgtggtaccaccactgccctgtctttgcacacatcagtcttcccggagtttagctcaaggaacgtgggtgggggtgctgtttgtgtttgtgtgtagtttttgccaatccaaacaaattggcaaagggggctggaagagtccatggtatttcccctatttgatttgcaatttgaactgtgcaaaaaataacgcgtgatttcatccctgtaccacgggacgaaggatcggctttgcatggtctggggaatcatggccccataccggagaagtaccgggcccacgcagccatgatccatccctcgagacggcccggcgacagcgtatccaacgtaggctcgggcccacgtcgcagtggaacaaagcccacagacacaaaaccccaccccccgctcgctgttgaactctctcttccctctctctctttctctctccaaaccccacgcacgccgcgtcgccgcctcctcctctccatctccgctgctattattgcccgcgcagacgcaggccaccatccttcctctcgctcacgctcgctgctatatgggggtcctcctcatcgcatcgcatcgcatcacctcgcacgggcgcgcgcgccgtgccgtgccgctagctcgatccgcctcgtacgccagctcgctcgctcgctcccccaccccgctgctgcacggctgcgcccgcgctgtcccctgtccccccgctcgccgcggcgatttatacccaccacgccccctgctgctgctataatgcccatga

SEQ ID NO: 5 - DNA, 888 bp, Triticum aestivum
atgggggagggcgcggtggtggtgctggaggcgcccaagcccaggtcgccgccgaggtacccggacatgtgcggccgccggcgcctgcagctggaggtgcagatccttgaccgcgagctcacgttcctcaaggacgagctacatttacttgaaggggctcaaccagtctcacgttctggttgcttgaaagaggtaaacgagtttgtcggtacaaaacaagacccgctaataccaattaacaaaaggaagcaccggtcctgccgtctttattggtggatcagatcgaaactgtgcatatgtgcttcatggctgtgctgctcctgccaatgcctaccaacctgcaaaagaccaaggtgcttcgactgttcatgctgcgagccaaactgctcgtgctgcagcctgaactgctgcagctgcttcagtatcccttcgtgctgcaaaccaagctgtggctgctttgagtgctgcagctgcagcaaaccacagtgctgcagcagcggctgtaacccttgcggcgagtgcaagccggagtgcggctcgtgttccggcggcggctgctgcggcgacagcaggccaagctgcagctgctgcggcgagcagtgctgctcctgccctcgatgcacaggctgcttcagctgcttcaaggtccccaaatgctcgtgcgcgcagtgcttcaactgccagtcgtcgtgctgcaaggggcagccgtcgtgcttcaggtgccagtcgtcgtgctgcgacaagggaggctgctgcagcggcgggtcgtgcctgagctgcccgaagccgtcgtgcccggagtgctcctgcgggtgcgtgtggtcgtgcaaaaactgtacagacggatgccgatgcgcccggtgctgtgctggcgggtgcctgtgttaa

SEQ ID NO: 6 - DNA, 888 bp, Hordeum vulgare
atgggggagggcgcggtggtggtgctggagccgcccaagcccaggtcgccgccgaggtacccggacatgtgcggccgccggcgcctgcagctggaggtgcagatccttgaccgcgagctcacgttcctcaaggacgagctacatttacttgaaggggctcaaccagtctcacgttctgcttgcttgaaagaggtaaatgagtttgttggtacaaaacaagatccactgataccaattaacaaaaggaagcatcggtcctgccgtctttattggtggatcagatcaaaactgtgcgtatgtgcttcatggctgtgctgctcctgccaatgcctaccaacctgcaaaagaccaagctgcttggactgttcatgctgcgagccaaactgctcgtgctgcagcccgaactgctgcagctgcttcaagatcccttcgtgctgcaaaccgagctgcggctgcttcgggtgctgcagctgcagcaaaccacagtgctgcagcggcggctgtaacccttgcggcgagtgcaagccggagtgtggctcttgttccgccggtggctgctgcggcgactgcaagccaagctgcagctgctgcggcgagcagtgccagtgctgctcctgccctcgatgcacggggggctgcttcaagctcccgaaatgctcgtgcgcgcagtgcttcaactgccagtcgtcgtgctgcaaggggcagccgtcgtgcttcaggtgccagtcgtcgtgctgcgacaagggcggctgctgcagcagcgggtcgtgcctgagctgcccgaagccgtcgtgcccggagtgctcctgcgggtgcgtgtggtcgtgcaaaaactgtacagacggatgccgatgcgcccggtgctgtgctagcgggtgcctgtgttga

SEQ ID NO: 7 - DNA, 1353 bp, Zea mays
atggcggcaccgggaagcccgacgccgcgccggctcccacgcccgcgccaacgccgctgccgccagaaaagcctctcccgggagagtgctgcggcagcgggtgcgttcgctgcgtcggggacatatatttcgacgagctcgacgcgtacgacaaggccctcgccgcgcgcgcggcctcctcaggctccggcggcaaggacgactctgctgataccaagcccaaagaaggtgccaaatcctgaagtgcgcctctcatgtgacgagctacaattacttgaaggagttccaccagtttctagaagttgtaaagaggtaaatgactttgttggtactaaacaagacccactaataccgataacgaaraagacacacagatcatgccgtcttttttggtggatcagatcaaaactgtgcatatgcgtgccatggttctgctgttcctgccattgcrtgcccaastgcaaaaggccatgcttcttggattgctcctgctgctcatgcccagatctgtcatgctgctatcccagctgcaaatcatgcaacaaaccatgctttgggcccaacagctgctcatgttgcgacatatcatgccgcaagccagattgcccatcctgtacctcgagctgcagctcgtgctgcaatcggccatgtggcaacccagactgcagctcgtgctgcacctgcaatccatcatgctgtaaaccaagctgcaactcgtgctgcagaccaaactgtagctcgtgctgcaatctgtcatgctgcaaacccagctgcagctcatgcttctcatgctgtaaagcgaactccagctcatgctgtaaacccaaatgcagctgcttcaaggccctctcgtgctgcaaattacagtgcagcccaaactgctgcacttgcagtctcccgagctgctcaggctgcaacccctgcggcagttgcaagcaatgctgctcatgccccagcgactgctttgattgcaagccaaactgcggctgtttcggcgcgcaatgctgcagctgcgtgggatgctgctcgtgctcctgccctcggtgctcaagctgcttcggttgcttcaagtacttcaagtgctccaacctgttcgggtgctgctcctgcaagcagtgcttcaagtgccagtcctcgtgctgcaagggggcgccgtcctgctgcaaatgccagtcgtcgtgctgtgagggtgaagacggcagcagcagctgctggaggtcatgctgcagcgttccgaagccggattgccctgggtgttcgtgcgggtgcgtttggtcctgcaggaagtgtacagaagggtgccgatgttctgggtgccgcaaaccatgctgtgccaccggatgcttgtgctga

SEQ ID NO: 8 - DNA, 828 bp, Zea mays
atgggggaagcgccgcagcccaggtcgccgccgaggtacccggacctgtgcggccgccgccgcctgcagctcgaggtgcagatcctcaaccgggaggtcggcttcctcgagcgtcacgagtttgtcagtgcaaagaccgacccaatgataccagtaagcaaaaggagacatggatcttgcagcttctcgcggtggatcagatcaaaattgcgcacatgcttttcatgcctgtgctgctggtgccactgcttgccgaagcccaatgcgccaagctgcttcagctgttcttgttgcacctgccgcgacacgcagtgctgtactccgatctgccgctgttcaaagaccccttcgtgcagccccggctgctgcacctgcagccttcccagctgcagctgcaagacgccaccaggctgcggccattgccgaccccagtgcagcagctgctgctctagtggttgcagctgcgcggactgcccgtgctcctgctcctgccccccgtgctgcagctgccccggcttcttctcctgtgaggggtgctcggctggctgcctcggcgctctcaaccggtgtctcggcggcctgtcgtcgtgctgcagtgagatgcgaccttcctgctgcaagtgccagtcgtcgtgctgcgaggggggttcgtcctgccgcggcaccggcacgggagcgtgctgccgcggttcgtgcctcggtgccccggcgtcgtcgtgcccggagtgctcctgcggctgcgtgtgctcctgctccaggtgcaaaggaggatgccgctgcccgtcctgtggtagtaatccctgctgtcccggtggatgcttgtgctag

SEQ ID NO: 9 - protein, 195 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNCSCSCPSCSSCCDTSCCKPSCTCFNI

SEQ ID NO: 10 - protein, 426 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCYCCKCSPKCKRPRCLNCSCSSCCDEPCCKPNCSACCAGSCCSPDCCSCCKPNCSCCKTPSCCKPNCSCSCPSCSSCCDTSCCKPSCTCFNIFSCFKSLYSCFKIPSCFKSQCNCSSPNCCTCTLPSCSCKGCACPSCGCNGCGCPSCGCNGCGCPSCGCNGCGLPSCGCNGCGSCSCAQCKPDCGSCSTNCCSCKPSCNGCCGEQCCRCADCFSCSCPRCSSCFNIFKCSCAGCCSSLCKCPCTTQCFSCQSSCCKRQPSCCKCQSSCCEGQPSCCEGHCCSLPKPSCPECSCGCVWSCKNCTEGCRCPRCRNPCCLSGCLC

SEQ ID NO: 11 - protein, 295 aa, Triticum aestivum
MGEGAVVVLEAPKPRSPPRYPDMCGRRRLQLEVQILDRELTFLKDELHLLEGAQPVSRSGCLKEVNEFVGTKQDPLIPINKRKHRSCRLYWWIRSKLCICASWLCCSCQCLPTCKRPRCFDCSCCEPNCSCCSLNCCSCFSIPSCCKPSCGCFECCSCSKPQCCSSGCNPCGECKPECGSCSGGGCCGDSRPSCSCCGEQCCSCPRCTGCFSCFKVPKCSCAQCFNCQSSCCKGQPSCFRCQSSCCDKGGCCSGGSCLSCPKPSCPECSCGCVWSCKNCTDGCRCARCCAGGCLC

SEQ ID NO: 12 - protein, 295 aa, Hordeum vulgare
MGEGAVVVLEPPKPRSPPRYPDMCGRRRLQLEVQILDRELTFLKDELHLLEGAQPVSRSACLKEVNEFVGTKQDPLIPINKRKHRSCRLYWWIRSKLCVCASWLCCSCQCLPTCKRPSCLDCSCCEPNCSCCSPNCCSCFKIPSCCKPSCGCFGCCSCSKPQCCSGGCNPCGECKPECGSCSAGGCCGDCKPSCSCCGEQCQCCSCPRCTGGCFKLPKCSCAQCFNCQSSCCKGQPSCFRCQSSCCDKGGCCSSGSCLSCPKPSCPECSCGCVWSCKNCTDGCRCARCCASGCLC

SEQ ID NO: 13 - protein, 450 aa, Zea mays
  misc_feature (151)..(151): Xaa can be any naturally occurring amino acid
MAAPGSPTPRRLPRPRQRRCRQKSLSRESAAAAGAFAASGTYISTSSTRTTRPSPRARPPQAPAARTTLLIPSPKKVPNPEVRLSCDELQLLEGVPPVSRSCKEVNDFVGTKQDPLIPITKKTHRSCRLFWWIRSKLCICVPWFCCSCHCXPXCKRPCFLDCSCCSCPDLSCCYPSCKSCNKPCFGPNSCSCCDISCRKPDCPSCTSSCSSCCNRPCGNPDCSSCCTCNPSCCKPSCNSCCRPNCSSCCNLSCCKPSCSSCFSCCKANSSSCCKPKCSCFKALSCCKLQCSPNCCTCSLPSCSGCNPCGSCKQCCSCPSDCFDCKPNCGCFGAQCCSCVGCCSCSCPRCSSCFGCFKYFKCSNLFGCCSCKQCFKCQSSCCKGAPSCCKCQSSCCEGEDGSSSCWRSCCSVPKPDCPGCSCGCVWSCRKCTEGCRCSGCRKPCCATGCLC

SEQ ID NO: 14 - protein, 275 aa, Zea mays
MGEAPQPRSPPRYPDLCGRRRLQLEVQILNREVGFLERHEFVSAKTDPMIPVSKRRHGSCSFSRWIRSKLRTCFSCLCCWCHCLPKPNAPSCFSCSCCTCRDTQCCTPICRCSKTPSCSPGCCTCSLPSCSCKTPPGCGHCRPQCSSCCSSGCSCADCPCSCSCPPCCSCPGFFSCEGCSAGCLGALNRCLGGLSSCCSEMRPSCCKCQSSCCEGGSSCRGTGTGACCRGSCLGAPASSCPECSCGCVCSCSRCKGGCRCPSCGSNPCCPGGCLC

SEQ ID NO: 15 - DNA, 23 bp, Oryza sativa
cttcaactgcctgcgagaccacc

SEQ ID NO: 16 - DNA, 23 bp, Oryza sativa
gcttgactgacataatgccgcta

SEQ ID NO: 17 - DNA, 22 bp, Oryza sativa
taagccgatgattactccagac

SEQ ID NO: 18 - DNA, 24 bp, Oryza sativa
gttcatttaaagaagtcctcaccg

SEQ ID NO: 19 - DNA, 36 bp, Artificial Sequence
  dep1 forward primer
gctctagagtcgactcaacataagcaaccactgaga

SEQ ID NO: 20 - DNA, 36 bp, Artificial Sequence
  dep1 reverse primer
gctctagagtcgacctagatgttgaagcaggtgcag

SEQ ID NO: 21 - DNA, 28 bp, Artificial Sequence
  Primer sequence
cggaattcgtctctcagtgagccgttcc

SEQ ID NO: 22 - DNA, 28 bp, Artificial Sequence
  Primer sequence
cgggatcctcatgggcattatagcagca

SEQ ID NO: 23 - DNA, 26 bp, Artificial Sequence
  Primer sequence
ctgcagtcgtaacccatgctgtctca

SEQ ID NO: 24 - DNA, 26 bp, Artificial Sequence
  Primer sequence
aagctttggcgagtaaatgagtccaa

SEQ ID NO: 25 - DNA, 26 bp, Artificial Sequence
  Primer sequence
gaattcgtctctcagtgagccgttcc

SEQ ID NO: 26 - DNA, 26 bp, Artificial Sequence
  Primer sequence
ggatcctcatgggcattatagcagca

SEQ ID NO: 27 - DNA, 32 bp, Artificial Sequence
  Primer sequence
cgggatccatgggggaggaggcggtggtgatg

SEQ ID NO: 28 - DNA, 28 bp, Artificial Sequence
  Primer sequence
gtcgactcaacataagcaaccactgaga

SEQ ID NO: 29 - protein, 80 aa, Oryza sativa
MAMAAAPRPKSPPAPPDPCGRHRLQLAVDALHREIGFLEGEINSIEGIHAASRCCREVDEFIGRTPDPFITISSEKRSHD

SEQ ID NO: 30 - protein, 426 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCCCCKCSPKCKRPRCLNCSCSSCCDEPCCKPNCSACCAGSCCSPDCCSCCKPNCSCCKTPSCCKPNCSCSCPSCSSCCDTSCCKPSCTCFNIFSCFKSLYSCFKIPSCFKSQCNCSSPNCCTCTHPSCSCKGCACPSCGCNGCGCPSCGCNGCGCPSCGCNGCGLPSCGCNGCGSCSCAQCKPDCGSCSTNCCSCKPSCNGCCGEQCCRCADCFSCSCPRSSSCFNIFKCSCAGCCSSLCKCPCTTQCFSCQSSCCKRQPSCCKCQSSCCEGQPSCCEGHCCSLPKPSCPECSCGCVWSCKNCTEGCRCPRCRNPCCLSGCLC

SEQ ID NO: 31 - protein, 426 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCCCCKCSPKCKRPRCLNCSCSSCCDEPCCKPNCSACCAGSCCSPDCCSCCKPNCSCCKTPSCCKPNCSCSCPSCSSCCDTSCCKPSCTCFNIFSCFKSLYSCFKIPSCFKSQCNCSSPNCCTCTHPSCSCKGCACPSCGCNGCGCPSCGCNGCGCPSYGCNGCGLPSCGCNGCGSCSCAQCKPDCGSCSTNCCSCKPSCNGCCGEQCCRCADCFSCSCPRSSSCFNIFKCSCAGCCSSLCKCPCTTQCFSCQSSCCKRQPSCCKCQSSCCEGQPSCCEGHCCSLPKPSCPECSCGCVWSCKNCTEGCRCPRCRNPCCLSGCLC

SEQ ID NO: 32 - protein, 426 aa, Oryza sativa
MGEEAVVMEAPRPKSPPRYPDLCGRRRMQLEVQILSREITFLKDELHFLEGAQPVSRSGCIKEINEFVGTKHDPLIPTKRRRHRSCRLFRWIGSKLCICISCLCCCCKCSPKCKRPRCLNCSCSSCCDEPCCKPNCSACCAGSCCSPDCCSCCKPNCSCCKTPSCCKPNCSCSCPSCSSCCDTSCCKPSCTCFNIFSCFKSLYNCFKIPSCFKSQCNCSSPNCCTCTHPSCSCKGCACPSCGCNGCGCPSCGCNGCGCPSCGCNGCGLPSCGCNGCGWCSCAQCKPDCGSCSTNCCSCKPSCNGCCGEQCCRCADCFSCSCPRSSSCFNIFKCSCAGCCSSLCKCPCTTQCFSCQSSCCKRQPSCCKCQSSCCEGQPSCCEGHCCSLPKPSCPECSCGCVWSCKNCTEGCRCPRCRNPCCLSGCLC

SEQ ID NO: 33 - protein, 283 aa, Triticum urartu
MGEGAVVVLEAPKPRSPPRYPDMCGRRRLQLEVQILDRELTFLKDELHLLEGAQPVSRSACLKEVNEFVGTKQDPLIPINKRKHRSCRLYWWIRSKLCICASWLCCSCQCLPTCKRPSCLDCSCCEPNCSCCSPNCRSCFKIPSCCSKPQCCSGGCNPCGECKPECGSCSAGGCCGDSRPSCSCCGEQCQCCSCPRCTGGCFKLPKCSCAQCFNCQSSCCKGQPSCFRCQSSCCDKGGCCSSGSCLSCPKPSCPECSCGCVWSCKNCTDGCRCARCCAGGCLC